(19) United States
(12) Patent Application Publication
(10) Pub. No.: US 2007/0167454 A1
(43) Pub. Date: Jul. 19, 2007

(54) Methods for treating hypertension

(76) Inventors: Christopher Lademacher, Evanston, IL (US); Lin Zhao, Vernon Hills, IL (US); Nancy Joseph-Ridge, Highwood, IL (US); Richard Johnson, Gainesville, FL (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 11/497,608

(22) Filed: Aug. 2, 2006

Related U.S. Application Data
(60) Provisional application No. 60/705,635, filed on Aug. 3, 2005.

(51) Int. Cl. A61K 31/53; A61K 31/426; A61K 31/4196; A61K 31/415

(52) U.S. Cl. 514/246; 514/365; 514/383; 514/406

(57) ABSTRACT

The present invention relates to methods of treating subjects suffering from pre-hypertension or hypertension by administering to a subject in need of treatment thereof a therapeutically effective amount of at least one xanthine oxidoreductase inhibiting compound or salt thereof.

### CROSS REFERENCE TO RELATED APPLICATIONS

This application claims priority to U.S. application Ser. No. 60/705,635, filed on Aug. 3, 2005, the contents of which are herein incorporated by reference.

### FIELD OF THE INVENTION

The present invention relates to methods of treating subjects suffering from pre-hypertension or hypertension. More specifically, the present invention involves administering to a subject in need of treatment thereof a therapeutically effective amount of at least one xanthine oxidoreductase inhibiting compound or salt thereof.

### BACKGROUND OF THE INVENTION

Blood pressure (hereinafter referred to as “BP”) is defined by a number of haemodynamic parameters taken either in isolation or in combination. Systolic blood pressure (hereinafter referred to as “SBP”) is the peak pressure exerted on the walls of the arteries during the contraction phase of the ventricles of the heart. Diastolic blood pressure (hereinafter referred to as “DBP”) is the minimum pressure exerted on the vessel walls when the heart muscle relaxes between beats and is filling with blood. The mean arterial blood pressure is the product of cardiac out put and peripheral vascular resistance.

Pre-hypertension has been defined as a SBP in the range of from 120 mmHg to 139 mmHG and/or a DBP in the range of from 80 mmHg to 89 mmHg. Pre-hypertension is considered to be a precursor of hypertension and a predictor of excessive cardiovascular risk (Julius, S., et al., N. Engl. J. Med., 354:1685-1697 (2006)).

Hypertension, or elevated BP, has been defined as a SBP of at least 140 mmHg and/or a DBP of at least 90 mmHg. By this definition, the prevalence of hypertension in developed countries is about 20% of the adult population, rising to about 60-70% of those aged 60 or more, although a significant fraction of these hypertensive subjects have normal BP when this is measured in a non-clinical setting. Some 60% of this older hypertensive population have isolated systolic hypertension, i.e. they have an elevated SBP and a normal DBP. Hypertension is associated with an increased risk of stroke, myocardial infarction, atrial fibrillation, heart failure, peripheral vascular disease and renal impairment (Fagard, RH; Am. J. Geriatric Cardiology, 11(1), 23-28 (2002); Brown, M J and Haycock, S; Drugs, 59(Suppl2), 1-12 (2000)).

The pathophysiology of hypertension is the subject of continuing debate. While it is generally agreed that hypertension is the result of an imbalance between cardiac output and peripheral vascular resistance, and that most hypertensive subjects have normal cardiac output and increased peripheral resistance there is uncertainty which parameter changes first (Beevers, G et al.; BMJ, 322, 912-916 (2001)).

U.S. Published Patent Application No. 2002/0019360 and its published PCT equivalent, WO 02/00210, describe methods of treating and preventing hypertension. The methods described in these publications involve administering a therapeutically effective amount of an agent capable of reducing uric acid levels to a patient in need of treatment thereof. Agents disclosed as being capable of reducing uric acid levels are: gene therapy agents, xanthine oxidase inhibitors, uricosuric agents, supplements of the uricase protein, urate channel inhibitors and combinations thereof. The only two xanthine oxidase inhibitors disclosed are allopurinol and carprofen.

Allopurinol has been used in the treatment of subjects suffering from gout. Structurally, allopurinol contains a purine ring. In terms of its function, allopurinol is known to have an effect, after administration to a subject in a therapeutically effective amount, on the activity of one or more enzymes involved in purine and pyrimidine metabolism. The enzymes involved in purine and pyrimidine metabolism include purine nucleotide phosphorylase and orotidine-5-monophosphate decarboxylase. Because of the effect allopurinol has on these enzymes, allopurinol is considered to be “non-selective” or “not selective” for these enzymes. Additionally, allopurinol is known to have a number of safety and side effects, including, vasculitis, angiitis, angioedema, cerebral vasculitis, arteritis, shock, toxic pustuloderma, granuloma annulare, rash, scaling eczema, Stevens-Johnson syndrome, toxic epidermal necrolysis, fever, acute gout (gouty flares), nausea, vomiting, diarrhea, abdominal discomfort, agranulocytosis, aplastic anemia, thrombocytopenia, eosinophilia, leucopenia, pure red cell aplasia, hepatitis, granulomatous hepatitis, hepatotoxicity, hepatic failure, hypersensitivity reactions (namely, the patient receiving treatment experiences one or more of the following, fever, leukocytosis, eosinophilia, lymphopenia, skin rashes, hepatomegaly, bronchospasm, rhinitis, shortness of breath, difficulty breathing, tightness in the chest and wheezing and elevated serum creatinine), aseptic meningitis, agitation, confusion, peripheral neuropathy, headache, paresthesia, catatonia, somnolence, ataxia, vertigo, peripheral axonal neuropathy with perforating foot ulcertation, macular eye lesions, macular retinitis, cataracts, cystitis, interstitial nephritis, acute tubular necrosis, nephrolithiasis, renal calculi, cystitis, angioedema and urolithiasis.

In contrast, carprofen is a well-known non-steroidal anti-inflammatory drug (hereinafter “NSAID”). NSAIDs are known to have a number of safety and side-effects, including, but not limited to, causing stomach ulceration (which can lead to performation and rupture of the stomach which is not only painful, but life-threatening), causing platelet deactivation (platelets should remain active for the purpose of controlling the ability to clot blood), causing decreased blood supply to the kidney (which could be cause a borderline patient to develop kidney failure) and may cause serious cardiovascular thrombotic events.

Despite the large number of drugs available in various pharmacological categories, including diuretics, alpha-adrenergic antagonists, beta-adrenergic antagonists, calcium channel blockers, angiotensin converting enzyme (hereinafter “ACE”) inhibitors and xanthine oxidase inhibitors containing a purine ring in their structure (such as allopurinol) and angiotensin receptor antagonists, the is still a need in the art for new and effective treatments of pre-hypertension and hypertension.

### SUMMARY OF THE PRESENT INVENTION

In one embodiment, the present invention relates to a method of treating pre-hypertension in a subject in need of treatment thereof. The method involves the step of administering to the subject a therapeutically effective amount of at least one compound, wherein said at least one compound is a xanthine oxidoreductase inhibitor or a pharmaceutically acceptable salt thereof. Examples of xanthine oxidoreductase inhibitors that can be used in the above-described method include, but are not limited to, 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-Cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (+), 3-(2-methyl-4-pyridyl)-5-cyano-4-isobutoxyphenyl)-1,2,4-triazole or pharmaceutically acceptable salts thereof. A subject receiving treatment for pre-hypertension pursuant to the above-described method has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg. Optionally, this method can further comprise administering to the subject a therapeutically effective amount of at least one anti-hypertensive compound with the at least one xanthine oxidoreductase inhibitor or pharmaceutically acceptable salt thereof.

In another embodiment, the present invention relates to a method of treating hypertension in a subject in need of treatment thereof. The method involves the step of administering to the subject a therapeutically effective amount of at least one compound, wherein said at least one compound is a xanthine oxidoreductase inhibitor or a pharmaceutically acceptable salt thereof. Examples of xanthine oxidoreductase inhibitors that can be used in the above-described method include, but are not limited to, 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-Cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (+), 3-(2-methyl-4-pyridyl)-5-cyano-4-isobutoxyphenyl)-1,2,4-triazole or pharmaceutically acceptable salts thereof. A subject receiving treatment for hypertension pursuant to the above-described method has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg. Optionally, this method can further comprise administering to the subject a therapeutically effective amount of at least one anti-hypertensive compound with the at least one xanthine oxidoreductase inhibitor or pharmaceutically acceptable salt thereof.

In yet another embodiment, the present invention relates to a method of lowering blood pressure in a subject. The method involves the step of administering to the subject a therapeutically effective amount of at least one compound, wherein said at least one compound is a xanthine oxidoreductase inhibitor or a pharmaceutically acceptable salt thereof. Examples of xanthine oxidoreductase inhibitors that can be used in the above-described method include, but are not limited to, 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-Cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole or pharmaceutically acceptable salts thereof. The at least one compound administered to the subject pursuant to this method can lower the systolic blood pressure, the diastolic blood pressure, the mean arterial pressure or a combination of the systolic blood pressure and diastolic blood pressure of the subject. A subject receiving treatment pursuant to the above-described method can have a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg. Alternatively, a subject receiving treatment pursuant to the above-described method can have a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg. Optionally, this method can further comprise administering to the subject a therapeutically effective amount of at least one anti-hypertensive compound with the at least one xanthine oxidoreductase inhibitor or pharmaceutically acceptable salt thereof.

In yet still another embodiment, the present invention relates to a method of decreasing pre-hypertension blood pressure or elevated blood pressure in a subject. The method involves the step of administering to the subject a therapeutically effective amount of at least one compound, wherein said at least one compound is a xanthine oxidoreductase inhibitor or a pharmaceutically acceptable salt thereof. Examples of xanthine oxidoreductase inhibitors that can be used in the above-described method include, but are not limited to, 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-Cyano-4-(2,2-dimethylpropoxy)phenyl]-lH-pyrazole-4-carboxylic acid, pyrazolo [1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole or pharmaceutically acceptable salts thereof. A subject being treated pursuant to this method can have a pre-hypertension blood pressure that comprises a systolic blood pressure in the range of 120 mmHg to 139 mmHg, a diastolic blood pressure in the range of 80 mmHg to 89 mmHg or a combination of a systolic blood pressure in the range of 120 mmHg to 139 mmHg and a diastolic blood pressure in the range of 80 mmHg to 89 mmHg. A subject being treated pursuant to this method can have an elevated blood pressure that comprises a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg. For example, the subject may have an elevated blood pressure comprising a systolic blood pressure of at least 160 mmHg or a diastolic blood pressure of at least 95 mmHg. The administration of the at least one compound pursuant to this method can lower the systolic blood pressure, the diastolic blood pressure, the mean arterial pressure or a combination of the systolic blood pressure and diastolic blood pressure of the subject. Optionally, this method can further comprise administering to the subject a therapeutically effective amount of at least one anti-hypertensive compound with the at least one xanthine oxidoreductase inhibitor or pharmaceutically acceptable salt thereof.

In still yet another embodiment, the present invention relates to a method of normalizing blood pressure in a subject having a history of pre-hypertension or hypertension. The method involves the step of administering to the subject a therapeutically effective amount of at least one compound, wherein said at least one compound is a xanthine oxidoreductase inhibitor or a pharmaceutically acceptable salt thereof. Examples of xanthine oxidoreductase inhibitors that can be used in the above-described method include, but are not limited to, 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-Cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (i), 3-(2-methyl-4-pyridyl)-5-cyano-4-isobutoxyphenyl)-1,2,4-triazole or pharmaceutically acceptable salts thereof. The administration of the at least one compound pursuant to the above described method can normalize the systolic blood pressure, the diastolic blood pressure, the mean arterial pressure or a combination of the systolic blood pressure and diastolic blood pressure of the subject. A subject receiving treatment pursuant to the above-described method can have a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg. Alternatively, a subject receiving treatment pursuant to the above-described method can have a systolic blood pressure of at least 140 mmHg, a diastolic blood pressure of at least 90 mmflg, a mean arterial pressure of at least 106 mmHg or a combination of a systolic blood pressure of at least 140 mmHg and a diastolic blood pressure of at least 90 mmHg. Optionally, this method can further comprise administering to the subject a therapeutically effective amount of at least one anti-hypertensive compound with the at least one xanthine oxidoreductase inhibitor or pharmaceutically acceptable salt thereof.

In yet another embodiment, the present invention relates to a method for treating pre-hypertension in a subject in need of treatment thereof. The method involves the step of administering to the subject an effective amount of at least one compound, wherein said at least one compound has the following formula:

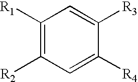

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;

wherein R₃ and R₄ are each independently a hydrogen or A, B, C or D as shown below:

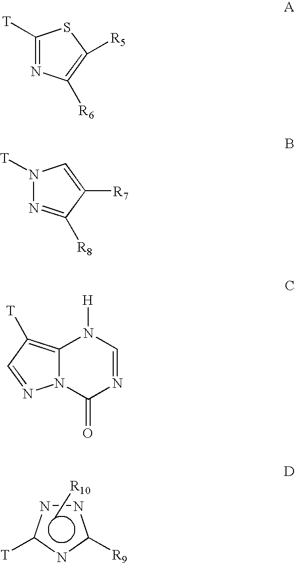

wherein T connects A, B, C or D to the aromatic ring shown above at R₁, R₂, R₃ or R₄.

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein Rg is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown in the above formula.

Examples of compounds having the above-identified formula that can be used in this method include, but are not limited to, 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-Cyano-4-(2,2-dimethylpropoxy)phenyl]-1H-pyrazole-4-carboxylic acid, pyrazolo [1,5-a]-1,3,5-triazin-4-(1H) one, 8-[3-methoxy-4-(phenylsulfinyl)phenyl]-sodium salt (±), 3-(2-methyl-4-pyridyl)-5-cyano-4-isobutoxyphenyl)-1,2,4-triazole or pharmaceutically acceptable salts thereof. A subject receiving treatment for pre-hypertension pursuant to the above-described method has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg. Optionally, this method can further comprise administering to the subject a therapeutically effective amount of at least one anti-hypertensive compound with the at least one compound or pharmaceutically acceptable salt thereof described above.

In yet another embodiment, the present invention relates to a method for treating hypertension in a subject in need of treatment thereof. The method involves the step of administering to the subject an effective amount of at least one compound, wherein said at least one compound has the following formula:

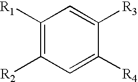

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;

wherein R₃ and R4 are each independently a hydrogen or A, B, C or D as shown below:

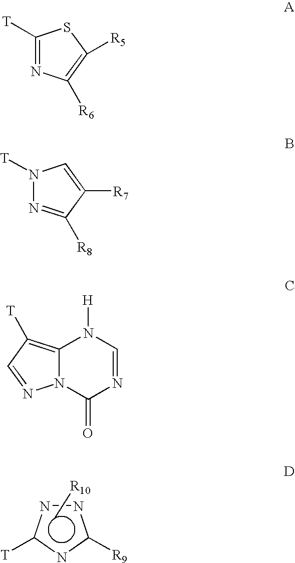

wherein T connects A, B, C or D to the aromatic ring shown above at R₁, R₂, R₃ or R₄.

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₉ is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown in the above formula.

Examples of compounds having the above-identified formula that can be used in this method include, but are not limited to, 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-Cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole or pharmaceutically acceptable salts thereof. A subject receiving treatment for hypertension pursuant to the above-described method has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg. Optionally, this method can further comprise administering to the subject a therapeutically effective amount of at least one anti-hypertensive compound with the at least one compound or pharmaceutically acceptable salt thereof described above.

In yet another embodiment, the present invention relates to a method of lowering blood pressure in a subject. The method involves the step of administering to the subject a therapeutically effective amount of at least one compound, wherein said at least one compound has the following formula:

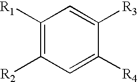

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;

wherein R₃ and R₄ are each independently a hydrogen or A, B, C or D as shown below:

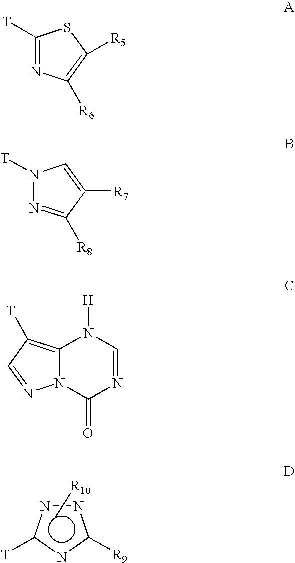

wherein T connects A, B, C or D to the aromatic ring shown above at R₁, R₂, R₃ or R₄.

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₉ is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown in the above formula.

Examples of compounds having the above-identified formula that can be used in this method include, but are not limited to, 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-Cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole or pharmaceutically acceptable salts thereof. The at least one compound administered to the subject pursuant to this method can lower the systolic blood pressure, the diastolic blood pressure, the mean arterial pressure or a combination of the systolic blood pressure and diastolic blood pressure of the subject. A subject receiving treatment pursuant to the above-described method can have a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg. Alternatively, a subject receiving treatment pursuant to the above-described method can have a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg. Optionally, this method can further comprise administering to the subject a therapeutically effective amount of at least one anti-hypertensive compound with the at least one compound or pharmaceutically acceptable salt thereof described above.

In yet still another embodiment, the present invention relates to a method of decreasing pre-hypertension blood pressure or elevated blood pressure in a subject. The method involves the step of administering to the subject a therapeutically effective amount of at least one compound, wherein said at least one compound has the following formula:

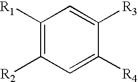

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;

wherein R₃ and R₄ are each independently a hydrogen or A, B, C or D as shown below:

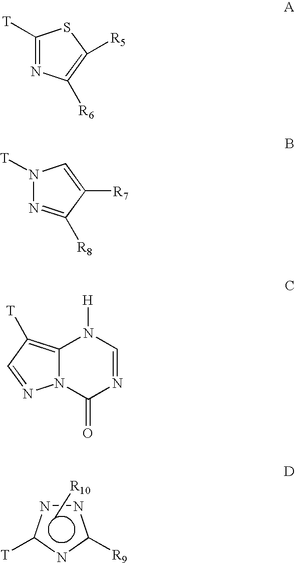

wherein T connects A, B, C or D to the aromatic ring shown above at R₁, R₂, R₃ or R₄.

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₉ is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown in the above formula.

Examples of compounds having the above-identified formula that can be used in this method include, but are not limited to, 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-Cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (+), 3-(2-methyl-4-pyridyl)-5-cyano-4-isobutoxyphenyl)-1,2,4-triazole or pharmaceutically acceptable salts thereof. A subject being treated pursuant to this method can have a pre-hypertension blood pressure that comprises a systolic blood pressure in the range of 120 mmHg to 139 mmHg, a diastolic blood pressure in the range of 80 mmHg to 89 mmHg or a combination of a systolic blood pressure in the range of 120 mmHg to 139 mmHg and a diastolic blood pressure in the range of 80 mmHg to 89 mmHg. A subject being treated pursuant to this method can have an elevated blood pressure that comprises a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg. For example, the subject may have an elevated blood pressure comprising a systolic blood pressure of at least 160 mmHg or a diastolic blood pressure of at least 95 mmHg. The administration of the at least one compound pursuant to this method can lower the systolic blood pressure, the diastolic blood pressure, the mean arterial pressure or a combination of the systolic blood pressure and diastolic blood pressure of the subject. Optionally, this method can further comprise administering to the subject a therapeutically effective amount of at least one anti-hypertensive compound with the at least one compound or pharmaceutically acceptable salt thereof described above.

In still yet another embodiment, the present invention relates to a method of normalizing blood pressure in a subject having a history of pre-hypertension or hypertension. The method involves the step of administering to the subject a therapeutically effective amount of at least one compound, wherein said at least one compound has the following formula:

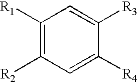

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;

wherein R₃ and R₄ are each independently a hydrogen or A, B, C or D as shown below:

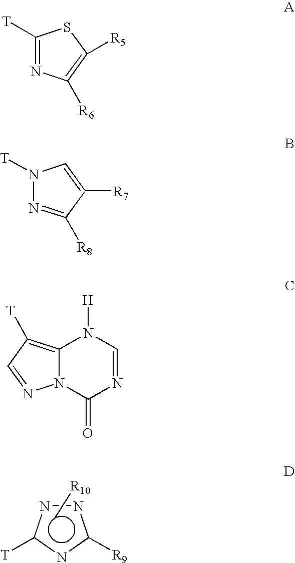

wherein T connects or attaches A, B, C or D to the aromatic ring shown above at R₁, R₂, R₃or R.

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₉ is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown in the above formula.

Examples of compounds having the above-identified formula that can be used in this method include, but are not limited to, 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-Cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole or pharmaceutically acceptable salts thereof. The administration of the at least one compound pursuant to the above described method can normalize the systolic blood pressure, the diastolic blood pressure, the mean arterial pressure or a combination of the systolic blood pressure and diastolic blood pressure of the subject. A subject receiving treatment pursuant to the above-described method can have a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg. Alternatively, a subject receiving treatment pursuant to the above-described method can have a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg. Optionally, this method can further comprise administering to the subject a therapeutically effective amount of at least one anti-hypertensive compound with the at least one compound or pharmaceutically acceptable salt thereof described above.

In yet another embodiment, the present invention relates to a method for treating pre-hypertension in a subject in need of treatment thereof. The method involves the step of administering to the subject an effective amount of at least one compound, wherein said at least one compound has the following formula:

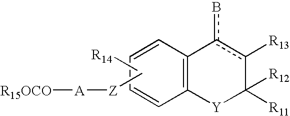

wherein R₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R₁₇′, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R₁₇ are each independently a hydrogen or a substituted or unsubstituted lower alkyl;

wherein R₁₅ is a hydrogen or a pharmaceutically active ester-forming group;

wherein A is a straight or branched hydrocarbon radical having one to five carbon atoms;

wherein B is a halogen, an oxygen, or a ethylenedithio;

wherein Y is an oxygen, a sulfur, a nitrogen or a substituted nitrogen;

wherein Z is an oxygen, a nitrogen or a substituted nitrogen; and

the dotted line refers to either a single bond, a double bond, or two single bonds.

A subject receiving treatment for pre-hypertension pursuant to the above-described method has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg. Optionally, this method can further comprise administering to the subject a therapeutically effective amount of at least one anti-hypertensive compound with the at least one compound or pharmaceutically acceptable salt thereof described above.

In yet another embodiment, the present invention relates to a method for treating hypertension in a subject in need of treatment thereof. The method involves the step of administering to the subject an effective amount of at least one compound, wherein said at least one compound has the following formula:

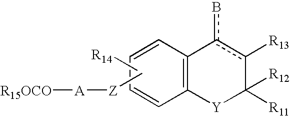

wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R₁₇′, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R₁₇′ are each independently a hydrogen or a substituted or unsubstituted lower alkyl;

wherein R₁₅ is a hydrogen or a pharmaceutically active ester-forming group;

wherein A is a straight or branched hydrocarbon radical having one to five carbon atoms;

wherein B is a halogen, an oxygen, or a ethylenedithio;

wherein Y is an oxygen, a sulfur, a nitrogen or a substituted nitrogen;

wherein Z is an oxygen, a nitrogen or a substituted nitrogen; and

the dotted line refers to either a single bond, a double bond, or two single bonds.

A subject receiving treatment for hypertension pursuant to the above-described method has a systolic blood pressure of at least 140 mmHg, a diastolic blood pressure of at least 90 mmHg, a mean arterial pressure of at least 106 mmHg or a combination of a systolic blood pressure of at least 140 mmflg and a diastolic blood pressure of at least 90 mmHg. Optionally, this method can further comprise administering to the subject a therapeutically effective amount of at least one anti-hypertensive compound with the at least one compound or pharmaceutically acceptable salt thereof described above.

In yet another embodiment, the present invention relates to a method of lowering blood pressure in a subject. The method involves the step of administering to the subject a therapeutically effective amount of at least one compound, wherein said at least one compound has the following formula:

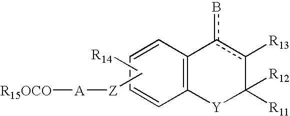

wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R₁₇′, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R₁₇ are each independently a hydrogen or a substituted or unsubstituted lower alkyl;

wherein R₁₅ is a hydrogen or a pharmaceutically active ester-forming group;

wherein A is a straight or branched hydrocarbon radical having one to five carbon atoms;

wherein B is a halogen, an oxygen, or a ethylenedithio;

wherein Y is an oxygen, a sulfur, a nitrogen or a substituted nitrogen;

wherein Z is an oxygen, a nitrogen or a substituted nitrogen; and

the dotted line refers to either a single bond, a double bond, or two single bonds.

The at least one compound administered to the subject pursuant to this method can lower the systolic blood pressure, the diastolic blood pressure, the mean arterial pressure or a combination of the systolic blood pressure and diastolic blood pressure of the subject. A subject receiving treatment pursuant to the above-described method can have a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg. Alternatively, a subject receiving treatment pursuant to the above-described method can have a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg. Optionally, this method can further comprise administering to the subject a therapeutically effective amount of at least one anti-hypertensive compound with the at least one compound or pharmaceutically acceptable salt thereof described above.

In yet still another embodiment, the present invention relates to a method of decreasing pre-hypertension blood pressure or elevated blood pressure in a subject. The method involves the step of administering to the subject a therapeutically effective amount of at least one compound, wherein said at least one compound has the following formula:

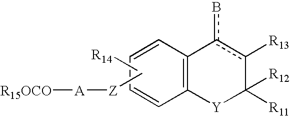

wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R₁₇′, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R₁₇′ are each independently a hydrogen or a substituted or unsubstituted lower alkyl;

wherein R₁₅ is a hydrogen or a pharmaceutically active ester-forming group;

wherein A is a straight or branched hydrocarbon radical having one to five carbon atoms;

wherein B is a halogen, an oxygen, or a ethylenedithio;

wherein Y is an oxygen, a sulfur, a nitrogen or a substituted nitrogen;

wherein Z is an oxygen, a nitrogen or a substituted nitrogen; and

the dotted line refers to either a single bond, a double bond, or two single bonds.

A subject being treated pursuant to this method can have a pre-hypertension blood pressure that comprises a systolic blood pressure in the range of 120 mmHg to 139 mmHg, a diastolic blood pressure in the range of 80 mmHg to 89 mmHg or a combination of a systolic blood pressure in the range of 120 mmHg to 139 mmHg and a diastolic blood pressure in the range of 80 mmHg to 89 mmHg. A subject being treated pursuant to this method can have an elevated blood pressure that comprises a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg. For example, the subject may have an elevated blood pressure comprising a systolic blood pressure of at least 160 mmHg or a diastolic blood pressure of at least 95 mmHg. The administration of the at least one compound pursuant to this method can lower the systolic blood pressure, the diastolic blood pressure, the mean arterial pressure or a combination of the systolic blood pressure and diastolic blood pressure of the subject. Optionally, this method can further comprise administering to the subject a therapeutically effective amount of at least one anti-hypertensive compound with the at least one compound or pharmaceutically acceptable salt thereof described above.

In still yet another embodiment, the present invention relates to a method of normalizing blood pressure in a subject having a history of pre-hypertension or hypertension. The method involves the step of administering to the subject a therapeutically effective amount of at least one compound, wherein said at least one compound has the following formula:

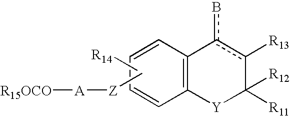

wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R₁₇′, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R₁₇′ are each independently a hydrogen or a substituted or unsubstituted lower alkyl;

wherein R₁₅ is a hydrogen or a pharmaceutically active ester-forming group;

wherein A is a straight or branched hydrocarbon radical having one to five carbon atoms;

wherein B is a halogen, an oxygen, or a ethylenedithio;

wherein Y is an oxygen, a sulfur, a nitrogen or a substituted nitrogen;

wherein Z is an oxygen, a nitrogen or a substituted nitrogen; and

the dotted line refers to either a single bond, a double bond, or two single bonds.

The administration of the at least one compound pursuant to the above described method can normalize the systolic blood pressure, the diastolic blood pressure, the mean arterial pressure or a combination of the systolic blood pressure and diastolic blood pressure of the subject. A subject receiving treatment pursuant to the above-described method can have a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg. Alternatively, a subject receiving treatment pursuant to the above-described method can have a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg. Optionally, this method can further comprise administering to the subject a therapeutically effective amount of at least one anti-hypertensive compound with the at least one compound or pharmaceutically acceptable salt thereof described above.

### BRIEF DESCRIPTION OF THE FIGURES

FIG. 1 shows the effect of febuxostat on plasma uric acid in normal and oxonic acid (hereinafter “OA”)-dosed rats.

FIG. 2 shows the effect of febuxostat on systolic blood pressure (by tail cuff) in normal and OA-dosed rats.

FIG. 3 shows the effect of febuxostat on mean arterial pressure (under anesthesia) in normal and OA-dosed rats.

FIG. 4 shows the effect of febuxostat on renal arteriolar area (hereinafter “AA”) in normal and OA-dosed rats.

FIG. 5 shows the effect of febuxostat on renal arteriolar media to lumen (hereinafter “M/L”) ratio in normal and OA-dosed rats.

### DETAILED DESCRIPTION OF THE PRESENT INVENTION

Introduction

As mentioned briefly above, the present invention relates to methods for treating pre-hypertension or hypertension in a subject in need of treatment thereof. In addition, the present invention also relates to methods of lowering blood pressure in a subject, methods of decreasing pre-hypertension blood pressure or elevated blood pressure in a subject and methods of normalizing blood pressure in a subject having a history of pre-hypertension or hypertension. The methods mentioned above will generally comprise administering to a subject in need of such therapy a therapeutically or prophylactically effective amount of at least one xanthine oxidoreductase inhibiting compound or salt thereof to said subject.

Definitions

The terms “administer”, “administering”, “administered” or “administration” refer to any manner of providing a drug (such as, a xanthine oxidoreductase inhibitor) to a subject or patient. Routes of administration can be accomplished through any means known by those skilled in the art. Such means include, but are not limited to, oral, buccal, intravenous, subcutaneous, intramuscular, by inhalation and the like.

As used herein, the term “antihypertensive compound or compounds” refers to one or more compounds that can reduce or lower blood pressure in a subject. Example of antihypertensive compounds include, but are not limited to, diuretics, beta adrenergic blockers, calcium channel blockers, angiotensin converting enzyme inhibitors, vasodilators, sympatholytic drugs, and angiotensin II receptor antagonists.

As used herein, the phrase “diastolic blood pressure” refers to the minimum pressure exerted on the vessel walls when the heart muscle relaxes between beats and is filling with blood. Diastolic blood pressure is usually the second or bottom number in a blood pressure reading. Methods for measuring diastolic blood pressure are well known to those skilled in the art.

As used herein, the term or phrase “hypertension” or “elevated blood pressure” refers to a systolic blood pressure in a subject of at least 140 mmHg, a diastolic blood pressure in a subject of at least 90 mmHg, a mean arterial pressure of at least 106 mmHg or a combination of a systolic blood pressure of at least 140 mmHg and a diastolic blood pressure of at least 90 mmHg in a subject. Preferably, “hypertension” or “elevated blood pressure” refers to a systolic blood pressure in a subject of at least 160 mmHg, a diastolic blood pressure of at least 95 mmHg or a combination systolic blood pressure of at least 160 mmHg and a diastolic blood pressure of at least 95 mmHg.

As used herein, the phrases “lowering blood pressure” or “lower blood pressure” refer to blood pressure in a subject that is reduced upon intake of a xanthine oxidoreductase inhibitor compound in accordance with the methods of the present invention. Any amount of blood pressure lowering is acceptable, as long as it is reduced by a statistically significant amount. As discussed previously herein, blood pressure is typically represented by systolic blood pressure and/or a diastolic blood pressure. Most frequently, blood pressure is represented as systolic blood pressure over diastolic blood pressure. Normal blood pressure in a human subject is a systolic blood pressure of below 120 mm Hg and a diastolic blood pressure of 70 mm Hg (120/70 mm Hg) on average, but normal for a subject, such as a human being, can vary with the height, weight, fitness level, health, emotional state, age, etc., of a subject. The xanthine oxidoreductase inhibitor compounds of the present invention can be used to lower blood pressure, such as systolic blood pressure, diastolic blood pressure, mean arterial pressure or a combination of systolic blood pressure and diastolic blood pressure by 1%, 2%, 3%, 4%, 5%, 6%, 7%, 8%, 9%, 10%, 11%, 12%, 13%, 14%, 15%, 16%, 17%, 18%, 19%, 20%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50% over the intial or baseline blood pressure taken in a subject.

As used herein, the phrase “mean arterial blood pressure” “mean arterial pressure” or “MAP” refer to the product of cardiac output and peripheral vascular resistance. MAP is used to assess the hemodynamic status of a patient. More specifically, it is considered the perfusion pressure seen by organs in the body. Formulas for approximating MAP are well known to those skilled in the art. An example of a formula that can be used to calculate MAP is:
MAP=⅔ diastolic blood pressure+⅓ systolic blood pressure

As used herein, the term “pharmaceutically acceptable” includes moieties or compounds that are, within the scope of sound medical judgment, suitable for use in contact with the tissues of humans and lower animals without undue toxicity, irritation, allergic response, and the like, and are commensurate with a reasonable benefit/risk ratio.

As used herein, the term “pre-hypertension” or “pre-hypertension blood pressure” refers to a systolic blood pressure in a subject in the range of 120 mmHg to 139 mmHg, a diastolic blood pressure in a subject in the range of 80 mmHg to 89 mmHg or a combination a systolic blood pressure in a subject in the range of 120 mmHg to 139 mmHg, a diastolic blood pressure in a subject in the range of 80 mmHg to 89 mmHg.

As used herein, the term “systolic blood pressure” refers to the peak pressure exerted on the walls of the arteries during the contraction phase of the ventricles of heart. Systolic blood pressure is usually the first or top number in a blood pressure reading. Methods for measuring systolic blood pressure are well known to those skilled in the art.

As used herein, the term “subject” refers to an animal, preferably a mammal, including a human or non-human. The terms patient and subject may be used interchangeably herein.

The terms “therapeutically effective amount” or “prophylactically effective amount” of a drug (namely, at least one xanthine oxidoreductase inhibitor or a salt thereof) refers to a nontoxic but sufficient amount of the drug to provide the desired effect. The amount of drug that is “effective” or “prophylactic” will vary from subject to subject, depending on the age and general condition of the individual, the particular drug or drugs, and the like. Thus, it is not always possible to specify an exact “therapeutically effective amount” or a “prophylactically effective amount”. However, an appropriate “therapeutically effective amount” or “prophylactically effective amount” in any individual case may be determined by one of ordinary skill in the art.

The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

As used herein, the term “xanthine oxidoreductase inhibitor” refers to any compound that (1) is an inhibitor of a xanthine oxidoreductase, such as, but not limited to, xanthine oxidase; and (2) chemically, does not contain a purine ring in its structure (i.e. is a “non-purine”). Examples of xanthine oxidoreductase inhibitors include, but are not limited to, 2-[4-(2-carboxypropoxy)-3-cyanophenyl]-4-methyl-5-thiazolecarboxylic acid and compounds having the following Formula I or Formula II:

Compounds of Formula I:

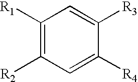

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;

wherein R₃ and R₄ are each independently a hydrogen or A, B, C or D as shown below:

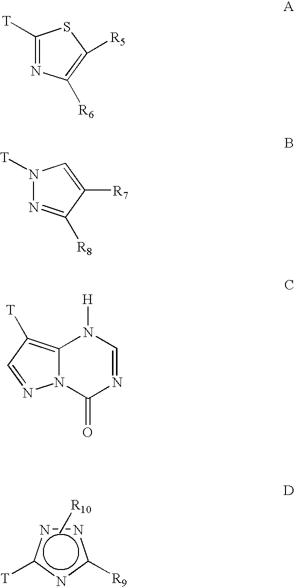

wherein T connects or attaches A, B, C or D to the aromatic ring shown above at R₁, R₂, R₃or R₄.

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₉ is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above in Formula I.

Compounds of Formula II:

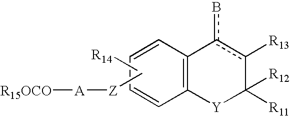

wherein R₁₁ and R₁₂ are each independently a hydrogen, a substituted or unsubstituted lower alkyl group, a substituted or unsubstituted phenyl (the substituted phenyl in this Formula II refers to a phenyl substituted with a halogen or lower alkyl, and the like. Examples include, but are not limited to, p-tolyl and p-chlorophenyl), or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl group, a substituted or unsubstituted phenyl (the substituted phenyl in this Formula II refers to a phenyl substituted with a halogen or lower alkyl group, and the like. Examples include, but are not limited to, p-tolyl and p-chlorophenyl), —OR₁₆ and —SO₂NR₁₇R₁₇′, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R₁₇ are each independently a hydrogen or a substituted or unsubstituted lower alkyl group;

wherein R₁₅ is a hydrogen or a pharmaceutically active ester-forming group;

wherein A is a straight or branched hydrocarbon radical having one to five carbon atoms;

wherein B is a halogen, an oxygen, or a ethylenedithio;

wherein Y is an oxygen, a sulfur, a nitrogen or a substituted nitrogen;

wherein Z is an oxygen, a nitrogen or a substituted nitrogen; and

the dotted line refers to either a single bond, a double bond, or two single bonds (for example, when B is ethylenedithio, the dotted line shown in the ring structure can be two single bonds).

As used herein, the term “lower alkyl(s)” group refers to a C₁-C₇ alkyl group, including, but not limited to, including methyl, ethyl, n-propyl, isopropyl, butyl, isobutyl, sec-butyl, tert-butyl, pentyl, isopentyl, hexyl, heptal and the like.

As used herein, the term “lower alkoxy” refers to those groups formed by the bonding of a lower alkyl group to an oxygen atom, including, but not limited to, methoxy, ethoxy, propoxy, isopropoxy, butoxy, isobutoxy, pentoxy, hexoxy, heptoxy and the like.

As used herein, the term “lower alkylthio group” refers to those groups formed by the bonding of a lower alkyl to a sulfur atom.

As used herein, the term “halogen” refers to fluorine, chlorine, bromine and iodine.

As used herein, the term “substituted pyridyl” refers to a pyridyl group that can be substituted with a halogen, a cyano group, a lower alkyl, a lower alkoxy or a lower alkylthio group.

As used herein, the term “four- to eight-membered carbon ring” refers to cyclobutyl, cyclopentyl, cyclohexyl, cycloheptyl, cyclooctyl and the like.

As used herein, the phrase “pharmaceutically active ester-forming group” refers to a group which binds to a carboxyl group through an ester bond. Such ester-forming groups can be selected from carboxy-protecting groups commonly used for the preparation of pharmaceutically active substances, especially prodrugs. For the purpose of the invention, said group should be selected from those capable of binding to compounds having Formula II wherein R₁₅ is hydrogen through an ester bond. Resultant esters are effective to increase the stability, solubility, and absorption in gastrointestinal tract of the corresponding non-esterified forms of said compounds having Formula II, and also prolong the effective blood-level of it. Additionally, the ester bond can be cleaved easily at the pH of body fluid or by enzymatic actions in vivo to provide a biologically active form of the compound having Formula II. Preferred pharmaceutically active ester-forming groups include, but are not limited to, 1-(oxygen substituted)-C₂ to C₁₅ alkyl groups, for example, a straight, branched, ringed, or partially ringed alkanoyloxyalkyl groups, such as acetoxymethyl, acetoxyethyl, propionyloxymethyl, pivaloyloxymethyl, pivaloyloxyethyl, cyclohexaneacetoxyethyl, cyclohexanecarbonyloxycyclohexylmethyl, and the like, C₃ to C₁₅ alkoxycarbonyloxyalkyl groups, such as ethoxycarbonyloxyethyl, isopropoxycarbonyloxyethyl, isopropoxycarbonyloxypropyl, t-butoxycarbonyloxyethyl, isopentyloxycarbonyloxypropyl, cyclohexyloxycarbonyloxyethyl, cyclohexylmethoxycarbonyloxyethyl, bomyloxycarbonyloxyisopropyl, and the like, C₂ to C₈ alkoxyalkyls, such as methoxy methyl, methoxy ethyl, and the like, C₄ to C₈ 2-oxacycloalkyls such as, tetrahydropyranyl, tetrahydrofuranyl, and the like, substituted C₈ to C₁₂ aralkyls, for example, phenacyl, phthalidyl, and the like, C₆ to C₁₂ aryl, for example, phenyl xylyl, indanyl, and the like, C₂ to C₁₂ alkenyl, for example, allyl, (2-oxo-1,3-dioxolyl)methyl, and the like, and [4,5-dihydro-4-oxo-1H-pyrazolo[3,4-d]pyrimidin-1-yl]methyl, and the like.

In R₁₆ in Formula II, the term “ester” as used in the phrase “the ester of carboxymethyl” refers to a lower alkyl ester, such as methyl or ethyl ester; and the term “ether” used in the phrase “the ether of hydroxyethyl” means an ether which is formed by substitution of the hydrogen atom of hydroxyl group in the hydroxyethyl group by aliphatic or aromatic alkyl group, such as benzyl.

The carboxy-protecting groups may be substituted in various ways. Examples of substituents include halogen atom, alkyl groups, alkoxy groups, alkylthio groups and carboxy groups.

As used herein, the term “straight or branched hydrocarbon radical” in the definition of A in Formula II above refers to methylene, ethylene, propylene, methylmethylene, or isopropylene.

As used herein, the substituent of the “substituted nitrogen” in the definition of Y and Z in Formula II above are hydrogen, lower alkyl, or acyl.

As used herein, the term “phenyl-substituted lower alkyl” refers to a lower alkyl group substituted with phenyl, such as benzyl, phenethyl or phenylpropyl.

The phrase “xanthine oxidoreductase inhibitor” as defined herein also includes metabolites, polymorphs, solvates and prodrugs of the compounds having the above described Formula I and Formula II. As used herein, the term “prodrug” refers to a derivative of the compounds shown in the above-described Formula I and Formula II that have chemically or metabolically cleavable groups and become by solvolysis or under physiological conditions compounds that are pharmaceutically active in vivo. Esters of carboxylic acids are an example of prodrugs that can be used in the dosage forms of the present invention. Methyl ester prodrugs may be prepared by reaction of a compound having the above-described formula in a medium such as methanol with an acid or base esterification catalyst (e.g., NaOH, H₂SO₄). Ethyl ester prodrugs are prepared in similar fashion using ethanol in place of methanol.

Examples of compounds having the above Formula I are: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-Cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (±) or 3-(2methyl-4-pyridyl)-5-cyano-4-isobutoxyphenyl)-1,2,4-triazole.

Preferred compounds having the above Formula I are: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid. These preferred compounds have also been found not have an effect at a therapeutically effective amount in a subject on the activity of any of the following enzymes involved in purine and pyrimidine metabolism: guanine deaminase, hypoxanthine-guanine phosphoribosyltransferse, purine nucleotide phosphorylase, orotate phosphoribosyltransferase or orotidine-5-monophosphate decarboxylase (i.e., meaning that it is “selective” for none of these enzymes which are involved in purine and pyrimidine metabolism). Assays for determining the activity for each of the above-described enzymes is described in Yasuhiro Takano, et al., Life Sciences, 76:1835-1847 (2005). These preferred compounds have also been referred to in the literature as nonpurine, selective inhibitors of xathine oxidase (NP/SIXO).

Examples of compounds having the above Formula II are described in U.S. Pat. No. 5,268,386 and EP 0 415 566 A1.

With the exception of pyrazolo [1,5-a]-1,3,5-triazin-4-(1H)-one, 8-[3-methoxy-4-(phenylsulfinyl)phenyl]-sodium salt (±), methods for making xanthine oxidoreductase inhibiting compounds of Formulas I and II for use in the methods of the present invention are known in the art and are described, for example, in U.S. Pat. Nos. 5,268,386, 5,614,520, 6,225,474, 7,074,816 and EP 0 415 566 A1 and in the publications Ishibuchi, S. et al., Bioorg. Med. Chem. Lett., 11:879-882 (2001) and which are each herein incorporated by reference. Other xanthine oxidoreductase inhibiting compounds can be found using xanthine oxidoreductase and xanthine in assays to determine if such candidate compounds inhibit conversion of xanthine into uric acid. Such assays are well known in the art.

Pyrazolo [1,5-a]-1,3,5-triazin-4-(1H)-one, 8-[3-methoxy-4-(phenylsulfinyl)phenyl]-sodium salt (±) is available from Otsuka Pharmaceutical Co. Ltd. (Tokyo, Japan) and is described in the following publications: Uematsu T., et al., “Pharmacokinetic and Pharmacodynamic Properties of a Novel Xanthine Oxidase Inhibitor, BOF-4272, in Healthy Volunteers, J. Pharmacology and Experimental Therapeutics, 270:453-459 (August 1994), Sato, S., A Novel Xanthine Deydrogenase Inhibitor (BOF-4272). In Purine and Pyrimidine Metabolism in Man, Vol. VII, Part A, ed. By P. A. Harkness, pp.135-138, Plenum Press, New York. Pyrazolo [1,5-a]-1,3,5-triazin-4-(1H)-one, 8-[3-methoxy-4-(phenylsulfinyl)phenyl]-sodium salt (±) can be made using routine techniques known in the art.

Description of the Invention

As mentioned briefly above, the present invention relates to methods of treating pre-hypertension, hypertension, lowering blood pressure and normalizing blood pressure in subjects in need of treatment thereof. The inventors of the present invention have discovered that a class of compounds known as xanthine oxidoreductase inhibitors can be used to treat pre-hypertension or hypertension, lower blood pressure and normalize blood pressure in said subjects.

The methods of the present invention involve establishing an initial or baseline blood pressure (such as a systolic blood pressure, a diastolic blood pressure, a mean arterial blood pressure or a combination of a systolic blood pressure and a diastolic blood pressure) for a subject. Methods for determining the blood pressure of a subject are well known in the art. For example, the systolic blood pressure and/or diastolic blood pressure of a subject can be determined using a sphygmomanometer (in mm of Hg) by a medical professional, such as a nurse or physician. Aneroid or electronic devices can also be used to determine the blood pressure of a subject and these devices and their use are also well known to those skilled in the art. Additionally, a 24-hour ambulatory blood pressure monitoring (hereinafter “ABPM”) device can be used to measure systolic blood pressure, diastolic blood pressure and heart rate. ABPM assesses systolic blood pressure, diastolic blood pressure and heart rate in predefined intervals (normally, the intervals are established at every 15 or 20 minutes, but any interval can be programmed) over a 24-hour period. The following parameters are then calculated from these readings after the data has been uploaded to a database. For example, ABPM can be used to measure the following: (1) the mean 24-hour systolic blood pressure of a subject; (2) the mean 24-hour diastolic blood pressure of a subject; (3) the mean daytime (The time period that constitutes “daytime” can readily be determined by those skilled in the art. For example, the “daytime” can be the time period from 6:00 a.m. until twelve noon or 7:00 a.m. to 10 p.m.) systolic blood pressure of a subject; (4) the mean daytime diastolic blood pressure of a subject; (4) the mean nighttime ((The time period that constitutes “nighttime” can readily be determined by those skilled in the art. For example, the “nightime” can be the time period from twelve midnight until 6:00 a.m. or 10:00 p.m. until 7:00 a.m.) systolic blood pressure of a subject; (5) the mean nighttime diastolic blood pressure of a subject; (6) the mean trough (The term “trough” refers to the time period at the end of the dosing period or the lowest point in drug levels and can readily be determined by those skilled in the art) systolic blood pressure of a subject; (7) the mean trough diastolic blood pressure of a subject; (8) the rate-pressure product (which is the product of heart rate and systolic blood pressure); and (9) the mean 24-hour mean rate-pressure product of a subject. The mean arterial pressure of a subject can be determined using a simple mathematical formula, such as the formula described previously herein (although alternative formulas are also known to those skilled in the art) once the systolic blood pressure and diastolic blood pressure of the subject has been determined. The time at which the blood pressure of the subject is determined is not critical for establishing the initial or baseline blood pressure reading. Once the initial or baseline blood pressure reading has been determined, a further determination is made by those skilled in the art as to whether or not the subject is suffering from (a) pre-hypertension or pre-hypertension blood pressure; or (b) hypertension or elevated blood pressure. For example, a baseline ABPM can be established 24-hours prior to beginning treatment of a subject in order to establish the initial or baseline ABPM in said subject. This initial or baseline APBM can also be used to determine whether or not the subject is suffering from pre-hypertension or hypertension.

Once a subject has been determined to be suffering from pre-hypertension (or pre-hypertension blood pressure) or hypertension (or elevated blood pressure), or if a subject has a history of suffering from pre-hypertension (or pre-hypertension blood pressure) or hypertension (or elevated blood pressure), the subject can be administered and thus treated with a therapeutically effective amount of at least one xanthine oxidoreductase inhibitor. Preferably, the subject ingests the at least one xanthine oxidoreductase inhibitor on a daily basis. After the subject has ingested the at least one xanthine oxidoreductase inhibitor for a specified period of time (such as a day, a week, two weeks, three weeks, four weeks, etc.), a second blood pressure reading is taken. This second blood pressure reading is compared to the initial or baseline blood pressure reading to determine whether there or not the subject exhibits a lower blood pressure (such as a lower systolic blood pressure, a lower diastolic blood pressure, a lower mean arterial pressure of a combination of a lower systolic blood pressure and a lower diastolic blood pressure). Any amount of statistically significant lower blood pressure (whether a statistically significant amount of a lower systolic blood pressure, a statistically significant amount of a lower diastolic blood pressure or a combination of a statistically significant amount of a lower systolic blood pressure and a lower diastolic blood pressure) is encompassed by the methods of the present invention. Moreover, the subject repeats the steps of ingesting the at least one xanthine oxidoreductase inhibitor (such as on a daily basis), taking a subsequent blood pressure reading at a specified period of time and comparing the subsequent blood pressure reading to the initial or baseline blood pressure reading, until a desirable level of blood pressure reduction (or lower blood pressure) has been achieved in the subject. Such a desirable level of blood pressure reduction can be determined by those skilled in the art. Such a desirable level of blood pressure reduction includes, but is not limited to, the normalization of the subject's blood pressure to a systolic blood pressure of below 120 mm Hg, a diastolic blood pressure of 70 mm Hg or a combination of a systolic blood pressure of below 120 mm Hg and a diastolic blood pressure of 70 mmHg. Additionally, once the subject has obtained a desirable level of blood pressure reduction, the subject can continue to take the at least one xanthine oxidoreductase inhibitor indefinitely in order to maintain said desired level of blood pressure reduction.

Because the xanthine oxidoreductase inhibitors of the present invention are effective in lowering blood pressure, these compounds can be used to treat subjects suffering from pre-hypertension (or pre-hypertension blood pressure) or hypertension (or elevated blood pressure). For example, the inventors discovered that in as little as four (4) weeks after beginning treatment with at least xanthine oxidoreductase inhibitor, patients suffering from hypertension exhibited a lower blood pressure (i.e., a statistically significant lower systolic blood pressure, a statistically significant lower diastolic blood pressure, a statistically significant lower mean arterial pressure or a combination of a statistically significant lower systolic blood pressure and a statistically significant lower diastolic blood pressure). Moreover, it is also believed that the xanthine oxidoreductase inhibitor compounds described herein can be used to further lower blood pressure in subjects already receiving one or more antihypertensive compounds. Thereupon, the xanthine oxidoreductase inhibitor compounds can be used as a monotherapy or as part of a combination therapy in lowering or decreasing blood pressure.

Compositions containing at least one xanthine oxidoreductase inhibitor in combination with at least one other pharmaceutical compound are contemplated for use in the methods of the present invention. Using the excipients and dosage forms described below, formulations containing such combinations are a matter of choice for those skilled in the art. Further, those skilled in the art will recognize that various coatings or other separation techniques may be used in cases where the combination of compounds are incompatible.

Compounds for use in accordance with the methods of the present invention can be provided in the form of pharmaceutically acceptable salts derived from inorganic or organic acids. Pharmaceutically acceptable salts are well-known in the art. For example, S. M. Berge et al. describe pharmaceutically acceptable salts in detail in J. Pharmaceutical Sciences, 66: 1 et seq. (1977). The salts can be prepared in situ during the final isolation and purification of the compounds or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, basic nitrogen-containing groups can be quatemized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

The at least one xanthine oxidoreductase inhibiting compound or salts thereof, may be formulated in a variety of ways that is largely a matter of choice depending upon the delivery route desired. For example, solid dosage forms for oral administration include capsules, tablets, pills, powders and granules. In such solid dosage forms, the xanthine oxidoreductase inhibiting compound may be mixed with at least one inert, pharmaceutically acceptable excipient or carrier, such as sodium citrate or dicalcium phosphate and/or a) fillers or extenders, such as, but not limited to, starches, lactose, sucrose, glucose, mannitol and silicic acid; b) binders, such as, but not limited to, carboxymethylcellulose, alginates, gelatin, polyvinylpyrrolidone, sucrose and acacia; c) humectants, such as, but not limited to glycerol; d) disintegrating agents, such as, but not limited to, agar-agar, calcium carbonate, potato or tapioca starch, alginic acid, certain silicates and sodium carbonate; e) solution retarding agents, such as, but not limited to, paraffin; f) absorption accelerators, such as, but not limited to, quaternary ammonium compounds; g) wetting agents, such as, but not limited to, cetyl alcohol and glycerol monostearate; h) absorbents, such as, but not limited to, kaolin and bentonite clay; and i) lubricants, such as, but not limited to, talc, calcium stearate, magnesium stearate, solid polyethylene glycols, sodium lauryl sulfate and mixtures thereof.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

Liquid dosage forms for oral administration include pharmaceutically acceptable emulsions, solutions, suspensions, syrups and elixirs. In addition to the xanthine oxidoreductase inhibiting compounds, the liquid dosage forms may contain inert diluents commonly used in the art such as, for example, water or other solvents, solubilizing agents and emulsifiers, such as, but not limited to, ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

The compositions can also be delivered through a catheter for local delivery at a target site, via an intracoronary stent (a tubular device composed of a fine wire mesh), or via a biodegradable polymer.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include, but are not limited to, water, ethanol, polyols (propylene glycol, polyethylene glycol, glycerol, and the like), vegetable oils (such as olive oil), injectable organic esters such as ethyl oleate, and suitable mixtures thereof.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form can be brought about by the use of agents delaying absorption, for example, aluminum monostearate and gelatin.

Suspensions, in addition to the active compounds (i.e., xanthine oxidoreductase inhibiting compounds or salts thereof), may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Proper fluidity can be maintained, for example, by the use of coating materials such as lecithin, by the maintenance of the required particle size in the case of dispersions and by the use of surfactants.

In some cases, in order to prolong the effect of the drug (i.e. xanthine oxidoreductase inhibiting compounds or salts thereof), it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microeneapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Dosage forms for topical administration of the compounds of this present invention include powders, sprays, ointments and inhalants. The active compound(s) is mixed under sterile conditions with a pharmaceutically acceptable carrier and any needed preservatives, buffers or propellants which can be required. Opthalmic formulations, eye ointments, powders and solutions are also contemplated as being within the scope of this invention.

It will be understood that formulations used in accordance with the present invention generally will comprise a therapeutically effective amount of one or more xanthine oxidoreductase inhibiting compounds. The phrase “therapeutically effective amount” or “prophylactically effective amount” as used herein means a sufficient amount of, for example, the composition, xanthine oxidoreductase inhibiting compound, or formulation necessary to treat the desired disorder, at a reasonable benefit/risk ratio applicable to any medical treatment. As with other pharmaceuticals, it will be understood that the total daily usage of a pharmaceutical composition of the invention will be decided by a patient's attending physician within the scope of sound medical judgment. The specific therapeutically effective or prophylactically effective dose level for any particular patient will depend upon a variety of factors including the disorder being treated and the severity of the disorder; activity of the specific compound employed; the specific composition employed; the age, body weight, general health, sex and diet of the patient; the time administration, route of administration, and rate of excretion of the specific compound employed; the duration of the treatment; drugs used in combination or coincidental with the specific compound employed; and other factors known to those of ordinary skill in the medical arts. For example, it is well within the skill of the art to start doses of the compound at levels lower than required to achieve the desired therapeutic effect and to gradually increase the dosage until the desired effect is achieved.

Formulations of the present invention are administered and dosed in accordance with sound medical practice, taking into account the clinical condition of the individual patient, the site and method of administration, scheduling of administration, and other factors known to medical practitioners.

Therapeutically effective or prophylactically effective amounts for purposes herein thus can readily be determined by such considerations as are known to those skilled in the art. The daily therapeutically effective or prophylactically effective amount of the xanthine oxidoreductase inhibiting compounds administered to a patient in single or divided doses range from about 0.01 to about 750 milligram per kilogram of body weight per day (mg/kg/day). More specifically, a patient may be administered from about 5.0 mg to about 300 mg once daily, preferably from about 20 mg to about 240 mg once daily and most preferably from about 40 mg to about 120 mg once daily of xanthine oxidoreductase inhibiting compounds.

By way of example, and not of limitation, examples of the present invention will now be given.

### EXAMPLE 1

A total of 103 subjects (9 in the placebo group, 26 in each the 2-[3-cyano-4-(2-methylpropoxy)phenyl]-4-methylthiazole-5-carboxylic acid (hereinafter referred to as “febuxostat”) 80 mg and 120 mg once daily (hereinafter referred to as “QD”) groups, 10 in the febuxostat 240 mg QD group and 32 in the 4-hydroxy-3,4-pyrazolopyrimidine (hereinafter referred to as “allopurinol”) 300/100 mg QD group), having a systolic BP≧160 mmHg or diastolic BP≧95 mmHg, and thus considered to have “elevated blood pressure”, were examined. Allopurinol is not a xanthine oxidoreductase inhibitor. Unlike xanthine oxidoreductase inhibitors, allopurinol contains a purine ring and also has an effect at a therapeutically effective amount in a subject on the activity of several enzymes involved in purine and pyrimidine metabolism, such as purine nucleotide phosphorylase or orotidine-5-monophosphate decarboxylase.

None of the above subjects were taking any antihypertensive agents at the baseline (start) of the study. These 104 subjects were part of two (2) double-blind (hereinafter referred to as “DB”) studies. One study was of 28 weeks in duration. During this time, the subjects received 80 mg, 120 mg or 240 mg QD of febuxostat, placebo or allopurinol 300 or 100 mg QD, depending on the subject's renal function. The second study was 52 weeks in duration. During this time, the subjects received 80 mg or 120 mg QD of febuxostat or allopurinol 300 mg QD.

Of these 103 subjects, all completed 4 weeks of treatment and 70 subjects completed 28 weeks of treatment. A total of 52 weeks of treatment was completed by 7 subjects in the febuxostat 80 mg QD group, 4 in the febuxostat 120 mg QD group and 14 in the allopurinol 300/100 mg QD group. Because of the shorter duration of one of the two DB studies, no subject in the placebo or in the febuxostat 240 mg QD groups was treated for the 52 weeks.

In the subjects, after 4 weeks of treatment, the mean change from baseline for systolic BP was −6.2 mmHg in the placebo group, −8.2 mmHg in the febuxostat 80 mg QD group, −11.0 mmHg in the febuxostat 120 mg QD group, −10.0 mmHg in the febuxostat 240 mg QD group and −7.7 mmHg in the allopurinol 300/100 mg QD group. The changes from baseline were statistically significant within the febuxostat 80 mg QD and 120 mg QD groups and the allopurinol 300/100 mg QD group. After 4 weeks of treatment, the mean change from baseline for diastolic BP was -3.3 mmHg in the placebo group, −3.7 mmHg in the febuxostat 80 mg QD group, −8.4 mmHg in the febuxostat 120 mg QD group, −8.9 mmHg in the febuxostat 240 mg QD group and −6.3 mmHg in the allopurinol 300/100 mg QD group. The changes from baseline were statistically significant within the febuxostat 120 mg QD and 240 mg QD groups and the allopurinol 300/100 mg QD group. After 4 weeks of treatment, the mean change from baseline for mean arterial BP was −4.3 mmHg in the placebo group, −5.2 mmHg in the febuxostat 80 mg QD group, −9.3 in the febuxostat 120 mg QD group, −9.3 mmHg in the febuoxstat 240 mg QD group and −6.8 mmHg in the allopurinol 300/100 mg QD group. The changes from baseline were statistically significant within the febuxostat 80 mg QD, 120 mg QD, 240 mg QD groups and the allopurinol 300/100 mg QD group.

In the subjects, after 28 weeks of treatment, the mean change from baseline for systolic BP was −4.3 mmHg in the placebo group, −13.0 mmHg in the febuxostat 80 mg QD group, −14.2 mmHg in the febuxostat 120 mg QD group, −8.0 mmHg in the febuxostat 240 mg QD group and −7.0 mmHg in the allopurinol 300/100 mg QD group. The changes from baseline were statistically significant within the febuxostat 80 mg QD and 120 mg QD groups and the allopurinol 300/100 mg QD group. After 28 weeks of treatment, the mean change from baseline for diastolic BP was −1.7 mmHg in the placebo group, −10.2 mmHg in the febuxostat 80 mg QD group, −6.4 mmHg in the febuxostat 120 mg QD group, −5.0 mmHg in the febuxostat 240 mg QD group and −8.2 mmHg in the allopurinol 300/100 mg QD group. The changes from baseline were statistically significant within the febuxostat 80 mg QD and 120 mg QD groups and the allopurinol 300/100 mg QD group. After 28 weeks of treatment, the mean change from baseline for mean arterial BP was −2.6 mmHg in the placebo group, −11.1 mmHg in the febuxostat 80 mg QD group, −9.0 in the febuxostat 120 mg QD group, −6.0 mmHg in the febuxostat 240 mg QD group and −7.8 mmHg in the allopurinol 300/100 mg QD group. The changes from baseline were statistically significant within the febuxostat 80 mg, 120 mg QD groups and the allopurinol 300/100 mg QD group.

In the subjects, after 52 weeks of treatment, the mean change from baseline for systolic BP was −13.4 mmHg in the febuxostat 80 mg QD group, −25.8 mmHg in the febuxostat 120 mg QD group and −9.4 mmHg in the allopurinol 300/100 mg QD group. The changes from Baseline were statistically significant within the febuxostat 80 mg QD and the allopurinol 300/100 mg QD group. After 52 weeks of treatment, the mean change from baseline for diastolic BP −12.3 mmHg in the febuxostat 80 mg QD group, −10.0 mmHg in the febuxostat 120 mg QD group, −10.9 mmHg in the allopurinol 300/100 mg QD group. The changes from baseline were statistically significant within the febuxostat 80 mg QD and the allopurinol 300/100 mg QD group. After 52 weeks of treatment, the mean change from baseline for mean arterial BP was −12.7 mmHg in the febuxostat 80 mg QD group, −15.2 in the febuxostat 120 mg QD group and −10.4 mmHg in the allopurinol 300/100 mg QD group. The changes from baseline were statistically significant within the febuxostat 80 mg QD group and the allopurinol 300/100 mg QD group.

### EXAMPLE 2

A total of 158 subjects (11 in the placebo group, 46 in the febuxostat 80 mg QD group, 39 in the febuxostat 120 mg QD group, 15 in the febuxostat 240 mg QD group and 47 in the allopurinol 300/100 mg QD group), having a systolic BP≧160 mmHg or diastolic BP≧95 mmHg, and thus considered to have “elevated blood pressure”, were examined. None of these subjects were taking any angiotensin-coverting enzyme inhibitors, but might have been taking some other type of antihypertensive drug at the baseline (start) of the study. These 158 subjects were part of two (2) DB studies. One study was of 28 weeks in duration during which subjects received 80 mg, 120 mg or 240 mg QD of febuxostat or placebo or allopurinol 300 or 100 mg QD, depending on the subject's renal function. The second study was 52 weeks in duration during which subjects received 80 mg or 120 mg QD of febuxostat or allopurinol 300 mg QD.

Of these 158 subjects, all completed 4 weeks of treatment and 114 completed 28 weeks of treatment. A total of 52 weeks of treatment was completed by 15 subjects in the febuxostat 80 mg QD group, 9 in the febuxostat 120 mg QD group and 17 in the allopurinol 300/100 mg QD group. Because of the shorter duration of one of the two DB studies, no subject in the placebo and febuxostat 240 mg QD groups was treated for 52 weeks.

In the subjects, after 4 weeks of treatment, the mean change from baseline for systolic BP was −7.8 mmHg in the placebo group, −7.2 mmHg in the febuxostat 80 mg QD group, −8.3 mmHg in the febuxostat 120 mg QD group, −18.9 mmHg in the febuxostat 240 mg QD group and −6.6 mmHg in the allopurinol 300/100 mg QD group. The changes from baseline were statistically significant within all treatment groups. After 4 weeks of treatment, the mean change from baseline for diastolic BP was −2.7 mmHg in the placebo group, −4.7 mmHg in the febuxostat 80 mg QD group, −7.2 mmHg in the febuxostat 120 mg QD group, −9.3 mmHg in the febuxostat 240 mg QD group and −6.7 mmHg in the allopurinol 300/100 mg QD group. The changes from baseline were statistically significant within the febuxostat 80 mg QD, 120 mg QD and 240 mg QD groups and the allopurinol 300/100 mg QD group. After 4 weeks of treatment, the mean change from baseline for mean arterial BP was −4.4 mmHg in the placebo group, −5.5 mmHg in the febuxostat 80 mg QD group, −7.6 in the febuxostat 120 mg QD group, −12.5 mmHg in the febuoxstat 240 mg QD group and −6.7 mmHg in the allopurinol 300/100 mg QD group. The changes from baseline were statistically significant within the febuxostat 80 mg QD, 120 mg QD, 240 mg QD groups and the allopurinol 300/100 mg QD group.

In the subjects, after 28 weeks of treatment, the mean change from baseline for systolic BP was −8.0 mmHg in the placebo group, −10.4 mmHg in the febuxostat 80 mg QD group, −11.0 mmHg in the febuxostat 120 mg QD group, −18.8 mmHg in the febuxostat 240 mg QD group and −9.1 mmHg in the allopurinol 300/100 mg QD group. The changes from baseline were statistically significant within the febuxostat 80 mg QD, 120 mg QD and 240 mg QD groups and the allopurinol 300/100 mg QD group. After 28 weeks of treatment, the mean change from baseline for diastolic BP was −3.8 mmHg in the placebo group, −8.7 mmHg in the febuxostat 80 mg QD group, −7.5 mmHg in the febuxostat 120 mg QD group, −10.0 mmHg in the febuxostat 240 mg QD group and −10.1 mmHg in the allopurinol 300/100 mg QD group. The changes from baseline were statistically significant within the febuxostat 80 mg QD, 120 mg QD and 240 mg QD groups and the allopurinol 300/100 mg QD group. After 28 weeks of treatment, the mean change from baseline for mean arterial BP was −5.2 mmHg in the placebo group, −9.2 mmHg in the febuxostat 80 mg QD group, −8.7 in the febuxostat 120 mg QD group, −12.9 mmHg in the febuxostat 240 mg QD group and −9.8 mmHg in the allopurinol 300/100 mg QD group. The changes from baseline were statistically significant within the febuxostat 80 mg QD, 120 mg QD, 240 mg QD groups and the allopurinol 300/100 mg QD group.

In the subjects, after 52 weeks of treatment, the mean change from baseline for systolic BP was −9.5 mmHg in the febuxostat 80 mg QD group, −19.4 mmHg in the febuxostat 120 mg QD group and −9.5 mmHg in the allopurinol 300/100 mg QD group. The changes from baseline were statistically significant within all treatment groups. After 52 weeks of treatment, the mean change from baseline for diastolic BP −8.1 mmHg in the febuxostat 80 mg QD group, −7.2 mmHg in the febuxostat 120 mg QD group, −11.4 mmHg in the allopurinol 300/100 mg QD group. The changes from baseline were statistically significant within the febuxostat 80 mg QD group and the allopurinol 300/100 mg QD group. After 52 weeks of treatment, the mean change from baseline for mean arterial BP was −8.5 mmHg in the febuxostat 80 mg QD group, −11.3 in the febuxostat 120 mg QD group and −10.8 mmHg in the allopurinol 300/100 mg QD group. The changes from baseline were statistically significant within the febuxostat 80 mg QD, 120 mg groups and the allopurinol 300/100 mg QD group.

### EXAMPLE 3

A total of 187 subjects (13 in the placebo group, 52 in the febuxostat 80 mg QD group, 48 in the febuxostat 120 mg QD group, 15 in the febuxostat 240 mg QD group and 59 in the allopurinol 300/100 mg QD group), having a systolic BP≧160 mmHg or diastolic BP≧95 mmHg, and thus considered to have “elevated blood pressure”, were examined. None of these subjects were taking any angiotensin antagonists, but might have been taking some other type of antihypertensive drug at the baseline (start) of the study. These 187 subjects were part of two (2) DB studies. One study was of 28 weeks in duration during which subjects received 80 mg, 120 mg or 240 mg QD of febuxostat or placebo or allopurinol 300 or 100 mg QD, depending on the subject's renal function. The second study was of 52 weeks in duration during which subjects received 80 mg or 120 mg QD of febuxostat or allopurinol 300 mg QD.

Of these 187 subjects, all completed 4 weeks of treatment and 132 completed 28 weeks of treatment. A total of 52 weeks of treatment was completed by 15 subjects in the febuxostat 80 mg QD group, 11 in the febuxostat 120 mg QD group and 22 in the allopurinol 300/100 mg QD group. Because of the shorter duration of one of the 2 DB studies, no subject in the placebo and febuxostat 240 mg QD groups were treated for 52 weeks.

In the subjects, after 4 weeks of treatment, the mean change from baseline for systolic BP was −9.1 mmHg in the placebo group, −6.7 mmHg in the febuxostat 80 mg QD group, −8.5 mmHg in the febuxostat 120 mg QD group, −11.3 mmHg in the febuxostat 240 mg QD group and −7.0 mmHg in the allopurinol 300/100 mg QD group. The changes from baseline were statistically significant within all treatment groups. After 4 weeks of treatment, the mean change from baseline for diastolic BP was −5.8 mmHg in the placebo group, −3.1 mmHg in the febuxostat 80 mg QD group, −7.5 mmHg in the febuxostat 120 mg QD group, −9.1 mmHg in the febuxostat 240 mg QD group and −5.7 mmHg in the allopurinol 300/100 mg QD group. The changes from baseline were statistically significant within the febuxostat 80 mg QD, 120 mg QD and 240 mg QD groups and the allopurinol 300/100 mg QD group. After 4 weeks of treatment, the mean change from baseline for mean arterial BP was −6.9 mmHg in the placebo group, −4.3 mmHg in the febuxostat 80 mg QD group, −7.8 in the febuxostat 120 mg QD group, −9.8 mmHg in the febuxostat 240 mg QD group and −6.1 mmHg in the allopurinol 300/100 mg QD group. The changes from baseline were statistically significant within the placebo, febuxostat 80 mg QD, 120 mg QD, 240 mg QD groups and the allopurinol 300/100 mg QD group.

In the subjects, after 28 weeks of treatment, the mean change from baseline for systolic BP was −8.2 mmHg in the placebo group, −12.6 mmHg in the febuxostat 80 mg QD group, −12.8 mmHg in the febuxostat 120 mg QD group, −9.2 mmHg in the febuxostat 240 mg QD group and −9.0 mmHg in the allopurinol 300/100 mg QD group. The changes from baseline were statistically significant within the febuxostat 80 mg QD and 120 mg QD groups and the allopurinol 300/100 mg QD group. After 28 weeks of treatment, the mean change from Baseline for diastolic BP was −6.0 mmHg in the placebo group, −7.3 mmHg in the febuxostat 80 mg QD group, −8.5 mmHg in the febuxostat 120 mg QD group, −4.9 mmHg in the febuxostat 240 mg QD group and −8.7 mmHg in the allopurinol 300/100 mg QD group. The changes from baseline were statistically significant within the febuxostat 80 mg QD and 120 mg QD groups and the allopurinol 300/100 mg QD group. After 28 weeks of treatment, the mean change from baseline for mean arterial BP was −6.7 mmHg in the placebo group, −9.0 mmHg in the febuxostat 80 mg QD group, −9.9 in the febuxostat 120 mg QD group, −6.3 mmHg in the febuxostat 240 mg QD group and −8.8 mmHg in the allopurinol 300/100 mg QD group. The changes from baseline were statistically significant within the febuxostat 80 mg QD, 120 mg QD groups and the allopurinol 300/100 mg QD group.

In the subjects, after 52 weeks of treatment, the mean change from baseline for systolic BP was −17.9 mmHg in the febuxostat 80 mg QD group, −18.6 mmHg in the febuxostat 120 mg QD group and −10.0 mmHg in the allopurinol 300/100 mg QD group. The changes from baseline were statistically significant within all treatment groups. After 52 weeks of treatment, the mean change from baseline for diastolic BP −8.4 mmHg in the febuxostat 80 mg QD group, −7.4 mmHg in the febuxostat 120 mg QD group, −11.6 mmHg in the allopurinol 300/100 mg QD group. The changes from baseline were statistically significant within the febuxostat 80 mg QD group and the allopurinol 300/100 mg QD group. After 52 weeks of treatment, the mean change from baseline for mean arterial BP was −11.6 mmHg in the febuxostat 80 mg QD group, −11.1 in the febuxostat 120 mg QD group, and −11.1 mmflg in the allopurinol 300/100 mg QD group. The changes from baseline were statistically significant within the febuxostat 80 mg QD, 120 mg QD groups and the allopurinol 300/100 mg QD group.

### EXAMPLES 1-3

demonstrate that xanthine oxidoreductase inhibitors, such as febuxostat, exhibit a more pronounced or significant effect on lowering the systolic blood pressure within all treatment groups when compared to allopurinol.

### EXAMPLE 4

The purpose of this study was to examine the effect of febuxostat on hypertension in an oxonic acid (hereinafter “OA”)-induced hyperuricemic model in Sprague-Dawley rats. Oxonic acid is an uricase inhibitor and can be used to induce experimental hyperuricemia. Previous studies have demonstrated that this model results in systemic hypertension as well as glomerular hypertension with preglomerular arteriolopathy (See, Mazzali M, et al., Hypertension 38:1101-1106 (2001), Mazzali M, et al., Am J Physiol Renal Physiol 282:F991-F997 (2002), Sanchez-Lozada LG, et al., Am J Physiol Renal Physiol 283:F1105-F 1110 (2002) and Sanchez-Lozada LG, et al., Kidney Int 67:237-247 (2005)).

Materials and Methods. To produce hyperuricemia, rats received OA (Sigma, St Louis Mo., USA) 750 mg/kg body weight daily by gastric gavage. To investigate the treatment effect of febuxostat (also referred to herein as “Fx”), the drug was administered in drinking water at 50 mg/L (approximately 5-6 mg/kg/day) four weeks after OA dosing while the respective controls received 5.84 mg/L of NaCl in drinking water (to maintain a salt concentration equivalent to the Febuxostat-containing water). The following four groups (n=10-11) were included in the study: Group 1, normal control rats which received no treatment for eight weeks; Group 2, Normal+Febuxostat rats which received no treatment for four weeks and then were treated with febuxostat for four weeks from Weeks 5 to 8; Group 3, OA rats which received OA for eight weeks; and Group 4, OA+Febuxostat rats which received OA for eight weeks and febuxostat for four weeks from Weeks 5 to 8. Body weight, food and water intakes were measured daily. Systolic blood pressure, obtained in conscious rats by a tail cuff sphygmomanometer, and plasma uric acid were measured in all animals at baseline and at the end of four and eight weeks. A renal micropuncture procedure along with systemic arterial blood pressure monitoring under pentobarbital anesthesia were performed at the end of eight weeks followed by morphologic evaluation of the renal preglomerular microvasculature.

Micropuncture Procedure to Assess Glomerular Hemodynamics. Animals were anesthetized with pentobarbital sodium (30 mg/kg, i.p.) and placed on a thermoregulated table to maintain body temperature at 37° C. Trachea, jugular veins, femoral arteries and the left ureter were catheterized with polyethylene tubing (PE-240, PE-50 and PE-10). The left kidney was exposed, placed in a Lucite holder, sealed with agar and covered with Ringer's solution. Mean arterial pressure (hereinafter “MAP”) was monitored using a pressure transducer (Model p23 db; Gould, San Juan, PR) connected to the catheter in the femoral artery and recorded on a polygraph (Grass Instruments, Quincy, Mass., USA). Blood samples were taken periodically and replaced with blood from a donor rat. Rats were maintained under euvolemic conditions by infusion of 10 mL/kg of body weight of isotonic rat plasma during surgery, followed by an infusion of 25% polyfructosan at 2.2 mL/h (Inutest, Fresenius Kabi, Linz, Austria). After 60 min, five to seven samples of proximal tubular fluid were obtained to determine flow rate and polyfructosan concentrations. Intratubular pressure under free-flow (hereinafter “FF”) and stop-flow (hereinafter “SFP”) conditions and peritubular capillary pressure (hereinafter “Pc”) were measured in other proximal tubules with a servo-null device (Servo Nulling Pressure System; Instrumentation for Physiology and Medicine, San Diego, Calif., USA). Glomerular colloid osmotic pressure was estimated from protein concentrations obtained from blood of the femoral artery (hereinafter “Ca”) and surface efferent arterioles (hereinafter “Ce”). Polyfructosan was measured in plasma and urine samples by the anthrone-based technique of Davidson and Sackner (See, Davidson WD et al., J Lab Clin Med 62:351-356 (1963)). Plasma samples were deproteinated first with trichloroacetic acid. After centrifugation, the supernatant was used for polyfructosan measurement. Polyfructosan concentrations in plasma and urine samples were assessed by addition of anthrone reagent followed by incubation at 45° C. for 50 min and reading in a spectrophotometer set at wavelength of 620 nm. Concentrations were calculated by interpolating the absorbance values using a standard curve (0.01-0.05 mg/mL). Total glomerular filtration rate (hereinafter “GFR”) was calculated using the following formula: GFR=(U×V)/P, where U is the polyfructosan concentration in urine, V is urine flow rate, and P is the polyfructosan concentration in plasma.

The volume of fluid collected from individual proximal tubules was estimated from the length of the fluid column in a constant bore capillary tube of known internal diameter. The concentration of tubular polyfructosan was measured by the microfluorometric method of Vurek and Pegram (Vurek GG, et al., Ann Biochem 16:409-419 (1966)). In brief, tubular fluid samples were transferred with a 8-nL pipette into capillary cuvettes sealed at one end which contained 3 μL of dimedone reagent (100 mg dimedone in 10 mL 85% ortho-phosphoric acid). Each cuvette was sealed immediately after adding the samples. Cuvettes were centrifugated five times at maximum speed during five minutes in a hematocrit centrifuge and heated in a boiling water bath for 10 min. Fluorescence was measured at excitation and emission wavelengths of 355 and 400 nm, respectively, (luminescence spectrometer, Aminco-Bowman Series 2, USA), against the reagent blank as 0% and 10 mg/mL polyfructosan as 100%. For each cuvette, the fluorescence was calculated as the mean of four readings and was rotated arbitrarily between the readings. Polyfructosan concentration was calculated by interpolating the fluorescence values using a standard curve (0.5-2.5 mg/mL). Single nephron glomerular filtration rate (hereinafter “SNGFR”) was calculated using the formula: SNGFR=(TF/P)_{PF}×V, where PF is the concentration of polyfructosan in tubular fluid (hereinafter “TF”) and plasma (hereinafter “P”), and V is the tubular flow rate which is obtained by timing the collection of tubular fluid (Baylis C, et al., Am J Physiol 230:1148-1158 (1976)).

Protein concentration in afferent and efferent samples was determined according to the method of Viets et al. (See, Viets JW, et al., Anal Biochem 88:513-521 (1978)). In brief, 5 nL of serum was mixed with 5 μL of borate buffer solution containing Brij and mercaptoethanol in a 100 μL glass capillary tube. Additionally, 5 μL of o-phthalaldehyde (hereinafter “OPT”) reagent was added. The contents were mixed by centrifuging the capillary tube several times in a hematocrit centrifuge. Fluorescence was measured 30-60 min after centrifugation at excitation and emission wavelengths of 362 and 419 nm, respectively, in a luminescence spectrometer (Aminco-Bowman Series 2, USA). Protein concentration was calculated by interpolating the values of fluorescence obtained in the samples against a standard curve (0.2-1.0 mg/niL). MAP, GFR, glomerular capillary hydrostatic pressure (hereinafter “PGC”), single-nephron plasma flow (hereinafter “QA”), afferent (hereinafter “AR”), efferent (hereinafter “ER”) and total (hereinafter “TR”) resistances, and ultrafiltration coefficient (hereinafter “Kf”) were calculated using the following equations previously reported (See, Baylis C, et al., Am J Physiol 230:1148-1158 (1976)):
PGC=SFP+πa, where πa is the colloid osmotic pressure of plasma obtained from femoral artery blood;
QA=SNGFR/SNFF, where SNFF is the single nephron filtration fraction;
SNFF=1-(Ca/Ce);
AR=(MAP-PGC/GBF)×(7.962×10¹⁰), where GBF is glomerular blood flow;
GBF=QA/(1-Hct);
ER=(PGC-Pc/GBF-SNGFR)×(7.962×10¹⁰);
TR=AR+ER;
Kf=SNGFR/EFP, where EFP is effective filtration pressure; and,
EFP=[(PGC-πa-FF)+(PGC-πe-FF)]/2, where πe is plasma colloid osmotic pressure from blood obtained in surface efferent arterioles.

Evaluation. Food and water intake were determined daily. Systolic blood pressure (hereinafter “SBP”) was measured in conscious animals by a tail cuff sphygmomanometer using an automated system (XBP-100, Kent Scientific Co, USA). All animals were preconditioned for blood pressure measurements one week before each experiment. Plasma UA, insulin and triglycerides were quantified using commercial kits (Diagnostic Chemicals Ltd, USA; Crystalchem, USA and Spinreact, Spain, respectively).

Renal Histology and Quantification of Morphology. After the micropuncture study, kidneys were washed by perfusion with phosphate-buffered saline and then fixed with 4% paraformaldehyde. Renal biopsies were embedded in paraffin. Four-pm sections of fixed tissue were stained with periodic acid Schiff (hereinafter “PAS”) reagent. Arteriolar morphology was assessed by indirect peroxidase immunostaining for alpha smooth-muscle actin (DAKO Corp, Carpinteria, Calif., USA). Renal sections incubated with normal rabbit serum were used as negative controls for immunostaining against alpha smooth-muscle actin.

For each arteriole, the outline of the vessel and its internal lumen (excluding the endothelium) were generated using computer analysis to calculate the total medial area (outline-inline) in 10 arterioles per biopsy. The media/lumen ratio was calculated by the outline/inline relationship (See, Sanchez-Lozada LG, et al., Am J Physiol Renal Physiol 283:F1105-F1110, (2002) and Sanchez-Lozada LG, et al., Kidney Int 67:237-247 (2005)). Quantifications were performed blinded. Statistical Analysis. Values were expressed as mean±standard error of the mean (hereinafter “SEM”). In the study, values from the respective four treatment groups were analyzed by one-way analysis of variance (hereinafter “ANOVA”). When the ANOVA p value was <0.05, the following comparisons were made using Bonferroni multiple comparison test: Normal Control vs. Normal+Fx, Normal Control vs. OA, Normal Control vs. OA+Fx and OA vs. OA+Fx. Pair wise comparisons were performed using contrast within the framework of the statistical model.

The relationship between variables was assessed by correlation analysis.

Results.

Body weight, food and water intake. As shown in Table 1 below, body weight did not differ between the groups at any time point, although there was slightly greater % weight gain in the Normal Control rats over the duration of Week 4-8. Food and water intake was also similar between groups, although during the first week, the OA+Fx group drank slightly more water compared to Normal Control rats. All rats behaved normally and no side effects were observed.

TABLE 1

| Parameter | Time/Period | Normal Control | Normal + Fx | OA | OA + Fx |
|---|---|---|---|---|---|
| BW (gr) | Baseline | 319.9 ± 4.1 | 319.9 ± 3.0 | 332.7 ± 3.8 | 323.6 ± 3.2 |
| | End of Week 4 | 342.2 ± 5.9 | 359.3 ± 7.2 | 362.5 ± 7.7 | 345.3 ± 5.7 |
| | End of Week 8 | 375.8 ± 6.1 | 377.7 ± 7.5 | 385.4 ± 7.4 | 363.2 ± 5.7 |
| % BW gain | End of Week 4 | 6.9 ± 0.8 | 11.9 ± 1.8 | 8.9 ± 1.6 | 6.7 ± 1.7 |
| from baseline | End of Week 8 | 17.5 ± 0.9 | 18.7 ± 1.8 | 15.8 ± 1.5 | 12.3 ± 1.9 |
| % BW gain | End of Week 8 | 9.8 ± 0.5 | 6.6 ± 0.8* | 6.4 ± 0.8* | 5.2 ± 0.7* |
| from Week 4 | | | | | |
| Daily Food | Week 1 | 16.2 ± 0.6 | 18.3 ± 0.6 | 15.7 ± 1.0 | 15.1 ± 0.7 |
| intake (gr)¹ | Week 4 | 15.9 ± 0.4 | 16.0 ± 0.7 | 16.5 ± 0.3 | 17.4 ± 0.7 |
| | Week 8 | 17.3 ± 0.3 | 18.0 ± 0.4 | 18.5 ± 0.6 | 18.6 ± 0.4 |
| Daily Water | Week 1 | 28.9 ± 1.5 | 34.5 ± 2.4 | 35.1 ± 1.1 | 36.6 ± 2.2* |
| intake (mL)¹ | Week 4 | 33.9 ± 1.3 | 32.3 ± 1.4 | 33.8 ± 1.3 | 33.8 ± 2.4 |
| | Week 8 | 41.5 ± 0.9 | 39.7 ± 1.7 | 41.0 ± 0.9 | 42.6 ± 2.3 |

¹mean ± SEM was calculated from the average of daily food or water intake over one week from each animal.
*indicates significant difference from Normal Control group.

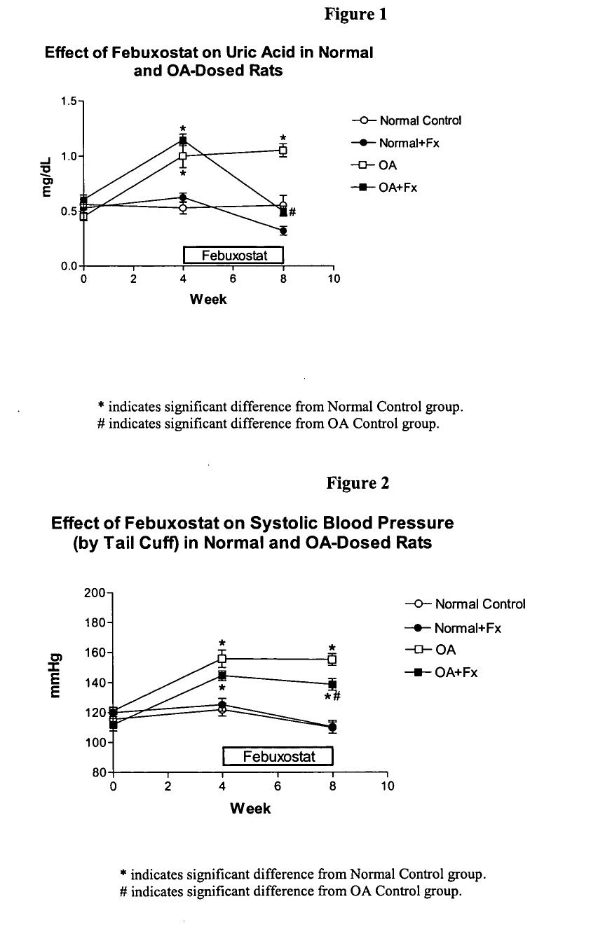

Plasma uric acid. Baseline values of plasma uric acid concentration were similar among all groups. With oxonic acid treatment rats showed a doubling in uric acid values at four weeks. The addition of febuxostat beginning at 4 weeks reduced the uric acid levels back into the normal range (See, FIG. 1). Normal rats receiving febuxostat had a decrease in uric acid levels to approximately 53% of control values, but this difference was not statistically significant.

Blood pressure. Systolic blood pressure data measured by the tail cuff method in conscious animals are shown in FIG. 2. All groups had similar baseline values. Oxonic acid treatment resulted in increased systolic pressure, which was present at both four and eight weeks. The addition of febuxostat to oxonic acid resulted in a significant but partial decrease in systolic BP. In contrast, febuxostat did not alter blood pressure in normal rats.

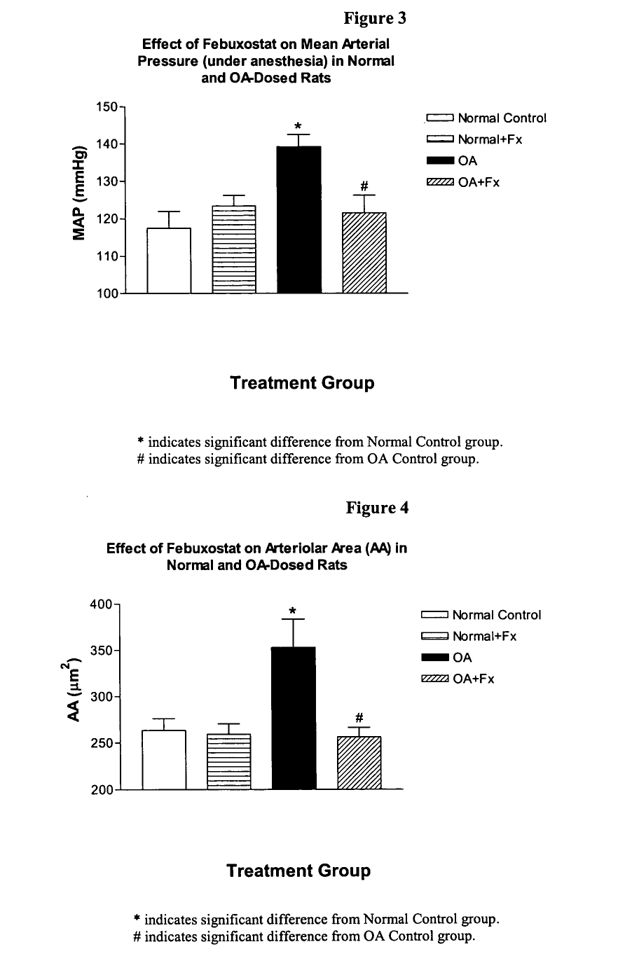

Mean arterial blood pressure (hereinafter “MAP”) was also measured at the end of the study by direct intra-arterial cannulation under anesthesia (See, FIG. 3). Oxonic acid treated rats had marked hypertension, and this was reduced to the normal range by febuxostat treatment (Normal Control: 118±4 mmHg; OA: 139±3 mmHg; OA+Fx: 122±5 mmHg) (FIG. 3). Febuxostat did not alter MAP in normal rats.

There was a significant positive correlation between uric acid concentrations at Week 8 and MAP when OA and OA+Fx rats were analyzed together (r=0.65, p=0.004). The correlation was also significant but weaker with systolic blood pressure measured by the tail cuff technique (r=0.46, p=0.04).

Glomerular hemodynamics. At the end of the eight weeks glomerular hemodynamics by the micropuncture technique were determined in all animals.

OA-treated rats had a significant elevation in glomerular capillary pressure (PGC), as noted by a rise in stop flow pressure (hereinafter “SFP”) (See, Table 2, below). Febuxostat treatment prevented these changes. When uric acid levels were correlated with PGC a significant correlation was found (r=0.74, p=0.0005, utilizing OA and OA+Fx groups). It has been previously reported that the increased glomerular pressure in hyperuricemic rats is mediated by an anomalous autoregulatory response of preglomerular vessels to the systemic hypertension (See, Sanchez-Lozada LG, et al., Am J Physiol Renal Physiol 283:F1105-F1110, (2002) and Sanchez-Lozada LG, et al., Kidney Int 67:237-247 (2005)). Consistent with this mechanism, at eight weeks we found a positive correlation between systemic arterial pressure and glomerular pressure [SBP (tail cuff) vs. PGC: r=0.59, p=0.01; MAP vs. PGC: r=0.76, p=0.0003]. An additional finding in OA+Fx rats was a numerically, but insignificantly higher ultrafiltration coefficient (hereinafter “Kf”) compared to the other groups. The Kf was also negatively correlated with uric acid levels when both OA and OA+Fx groups were analyzed (r=-0.53, p=0.02). Finally, febuxostat treatment did not alter glomerular hemodynamics in normal rats.

TABLE 2

| | Normal Control | Normal + FX | OA | OA + Fx |
|---|---|---|---|---|
| Parameter | (n = 8) | (n = 8) | (n = 8) | (n = 10) |
| Hct (%) | 0.47 ± 0.01 | 0.48 ± 0.01 | 0.48 ± 0.01 | 0.46 ± 0.01 |
| MAP (mmHg) | 118 ± 4 | 123 ± 3 | 139 ± 3* | 122 ± 5^{#} |
| GFR (mL/min) | 0.7 ± 0.1 | 0.9 ± 0.1 | 0.8 ± 0.1 | 1.0 ± 0.1 |
| SFP (mmHg) | 28.8 ± 1.2 | 30.7 ± 0.8 | 37.1 ± 1.2* | 29.7 ± 1.0^{#} |
| Pc (mmHg) | 13.2 ± 0.7 | 12.5 ± 0.8 | 12.9 ± 0.6 | 13.2 ± 0.7 |
| FF (mmHg) | 12.6 ± 0.8 | 13.4 ± 0.8 | 11.9 ± 0.8 | 12.9 ± 0.5 |
| PGC (mmHg) | 45.4 ± 1.7 | 46.3 ± 1.4 | 54.4 ± 1.5* | 46.5 ± 1.0^{#} |
| SNGFR (nL/min) | 35.0 ± 3.4 | 34.6 ± 2.3 | 51.2 ± 6.3 | 45.7 ± 5.2 |
| QA (nL/min) | 155.3 ± 18.3 | 135.0 ± 11.2 | 256.6 ± 49.2 | 183.6 ± 21.4 |
| AR (dyn · s · cm⁻⁵) | 2.6 ± 0.9 | 2.5 ± 0.2 | 1.9 ± 0.4 | 2.0 ± 0.3 |
| ER (dyn · s · cm⁻⁵) | 1.2 ± 0.3 | 1.3 ± 0.1 | 1.0 ± 0.2 | 1.0 ± 0.1 |
| TR (dyn · s · cm⁻⁵) | 3.8 ± 1.1 | 3.8 ± 0.3 | 2.9 ± 0.6 | 3.1 ± 0.4 |
| Kf (nL/s · mmHg) | 0.054 ± 0.009 | 0.046 ± 0.004 | 0.041 ± 0.005 | 0.070 ± 0.011 |

Hct: Hematocrit; MAP: mean arterial pressure; GFR: glomerular filtration rate; SFP: stop flow pressure; Pc: peritubular capillary pressure; FF: free flow tubular pressure; PGC: glomerular capillary pressure; SNGFR: single nephron GFR; QA: glomerular plasma flow; AR: afferent resistance; ER: efferent resistance; TR: total resistance; Kf: ultrafiltration coefficient.
*indicates significant difference from Normal Control group.
^{#}indicates significant difference from OA Control group.

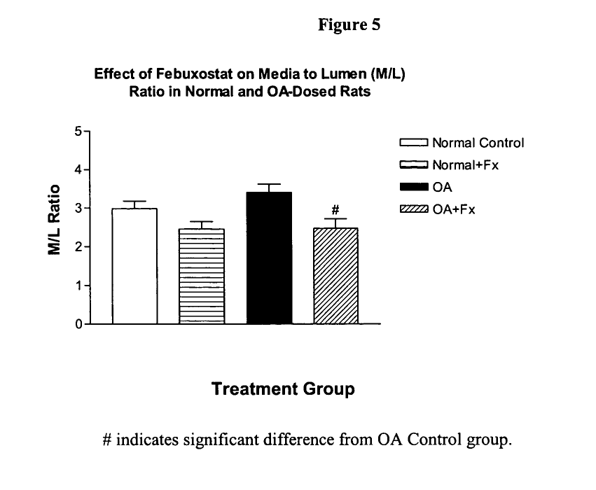

Renal arteriolar morphology. Oxonic acid treatment was associated with thickening of the afferent arteriole, as reflected by an increase in medial area (See, FIG. 4). Febuxostat treatment was able to alleviate this thickening (See, FIG. 4). A nonsignificant increase in media-lumen ratio was also observed with oxonic acid; this was significantly reduced by febuxostat (See, FIG. 5). Febuxostat had no effect on arteriolar morphology in normal rats.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

What is claimed is:

1. A method for treating pre-hypertension in a subject in need of treatment thereof, the method comprising the step of:
administering to the subject a therapeutically effective amount of at least one compound, wherein said at least one compound is a xanthine oxidoreductase inhibitor or a pharmaceutically acceptable salt thereof.

2. The method of claim 1, wherein the xanthine oxidoreductase inhibitor is selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole and a pharmaceutically acceptable salt thereof.

3. The method of claim 1, wherein the subject has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg.

4. A method for treating hypertension in a subject in need of treatment thereof, the method comprising the step of:
administering to the subject a therapeutically effective amount of at least one compound, wherein said at least one compound is a xanthine oxidoreductase inhibitor or a pharmaceutically acceptable salt thereof.

5. The method of claim 4, wherein the xanthine oxidoreductase inhibitor is selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo [1,5-a]-1,3 ,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole and a pharmaceutically acceptable salt thereof.

6. The method of claim 4, wherein the subject has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg.

7. A method of lowering blood pressure in a subject, the method comprising the step of:
administering to the subject a therapeutically effective amount of at least one compound wherein said at least one compound is a xanthine oxidoreductase inhibitor or a pharmaceutically acceptable salt thereof.

8. The method of claim 7, wherein the xanthine oxidoreductase inhibitor is selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole and a pharmaceutically acceptable salt thereof.

9. The method of claim 7, wherein the subject has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg.

10. The method of claim 7, wherein the subject has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg.

11. The method of claim 7, wherein the administration of the at least one compound lowers the systolic blood pressure, diastolic blood pressure, mean arterial pressure or a combination of the systolic blood pressure and diastolic blood pressure of the subject.

12. A method of decreasing pre-hypertension blood pressure or elevated blood pressure in a subject, the method comprising the step of:
administering to the subject a therapeutically effective amount of at least one compound, wherein said at least one compound is a xanthine oxidoreductase inhibitor or a pharmaceutically acceptable salt thereof.

13. The method of claim 12, wherein the xanthine oxidoreductase inhibitor is selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole and a pharmaceutically acceptable salt thereof.

14. The method of claim 12, wherein the subject has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg.

15. The method of claim 12, wherein the subject has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg.

16. The method of claim 12, wherein the subject has a systolic blood pressure of 160 mmHg or a diastolic blood pressure of at least 95 mmHg, or a combination of a systolic blood pressure of 160 mmHg and a diastolic blood pressure of at least 95 mmHg.

17. The method of claims 12 or 13, wherein the administration of the at least one compound lowers the systolic blood pressure, the diastolic blood pressure, the mean arterial pressure or a combination of the systolic blood pressure and the diastolic blood pressure of the subject.

18. A method of normalizing blood pressure in a subject having a history of pre-hypertension or hypertension, the method comprising the step of:
administering to the subject a therapeutically effective amount of at least one compound wherein said at least one compound is a xanthine oxidoreductase inhibitor or a pharmaceutically acceptable salt thereof.

19. The method of claim 18, wherein the xanthine oxidoreductase inhibitor is selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole and a pharmaceutically acceptable salt thereof.

20. The method of claim 18, wherein the administration of the at least one compound normalizes the systolic blood pressure, the diastolic blood pressure, the mean arterial pressure or a combination of the systolic blood pressure and diastolic blood pressure of the subject.

21. The method of claim 18, wherein the subject has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg.

22. The method of claim 18, wherein the subject has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg.

23. A method for treating pre-hypertension in a subject in need of treatment thereof, the method comprising the step of:
administering to the subject a therapeutically effective amount of a compound or a pharmaceutically acceptable salt thereof, wherein said compound comprises the formula: 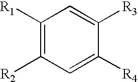
wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;
wherein R₃ and R₄ are each independently a hydrogen or A, B, C or D as shown below: 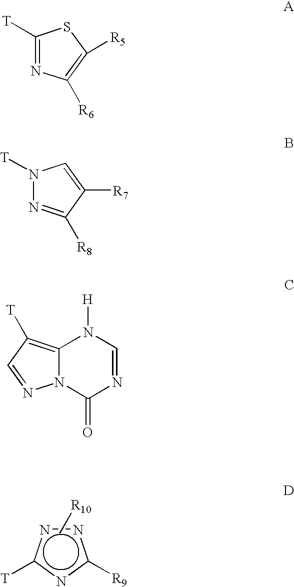
wherein T connects A, B, C or D to the aromatic ring shown above at R₁, R₂, R₃ or R₄.
wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₉ is an unsubstituted pyridyl group or a substituted pyridyl group; and
wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above.

24. The method of claim 23, wherein the compound is 2-[3-cyano-4-(2-methylpropoxy)phenyl]-4-methylthiazole-5-carboxylic acid or a pharmaceutically acceptable salt thereof.

25. The method of claim 23, wherein the compound is 2-[3-cyano-4-(3-hydroxy-2-methylpropoxy)phenyl]-4-methyl-5-thiazolecarboxylic acid or a pharmaceutically acceptable salt thereof.

26. The method of claim 23, wherein the compound is 2-[3-cyano-4-(2-hydroxy-2-methylpropoxy)phenyl]-4-methyl-5-thiazolecarboxylic acid or a pharmaceutically acceptable salt thereof.

27. The method of claim 23, wherein the compound is 2-(3-cyano-4-hydroxyphenyl)-4-methyl-5-thiazolecarboxylic acid or a pharmaceutically acceptable salt thereof.

28. The method of claim 23, wherein the compound is 2-[4-(2-carboxypropoxy)-3-cyanophenyl]-4-methyl-5-thiazolecarboxylic acid or a pharmaceutically acceptable salt thereof.

29. The method of claim 23, wherein the compound is 1-3-cyano-4-(2,2-dimethylpropoxy)phenyl]-1H-pyrazole-4-carboxylic acid or a pharmaceutically acceptable salt thereof.

30. The method of claim 23, wherein the compound is pyrazolo [1,5-a]-1,3,5-triazin-4-(1H)-one, 8-[3-methoxy-4-(phenylsulfinyl)phenyl]-sodium salt (±).

31. The method of claim 23, wherein the compound is 3-(2-methyl-4-pyridyl)-5-cyano-4-isobutoxyphenyl)-1,2,4-triazole or a pharmaceutically acceptable salt thereof.

32. The method of claim 23, wherein the subject has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg.

33. A method for treating hypertension in a subject in need of treatment thereof, the method comprising the step of:
administering to the subject a therapeutically effective amount of a compound or a pharmaceutically acceptable salt thereof, wherein said compound comprises the formula: 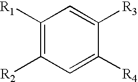
wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;
wherein R₃ and R4 are each independently a hydrogen or A, B, C or D as shown below: 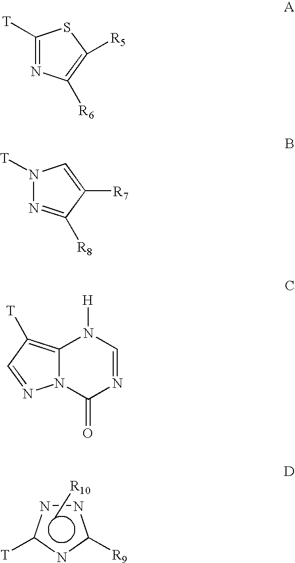
wherein T connects A, B, C or D to the aromatic ring shown above at R₁, R₂, R₃ or R4.
wherein R₅ and R6 are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₉ is an unsubstituted pyridyl group or a substituted pyridyl group; and
wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above.

34. The method of claim 33, wherein the compound is 2-[3-cyano-4-(2-methylpropoxy)phenyl]-4-methylthiazole-5-carboxylic acid or a pharmaceutically acceptable salt thereof.

35. The method of claim 33, wherein the compound is 2-[3-cyano-4-(3-hydroxy-2-methylpropoxy)phenyl]-4-methyl-5-thiazolecarboxylic acid or a pharmaceutically acceptable salt thereof.

36. The method of claim 33, wherein the compound is 2-[3-cyano-4-(2-hydroxy-2-methylpropoxy)phenyl]-4-methyl-5-thiazolecarboxylic acid or a pharmaceutically acceptable salt thereof.

37. The method of claim 33, wherein the compound is 2-(3-cyano-4-hydroxyphenyl)-4-methyl-5-thiazolecarboxylic acid or a pharmaceutically acceptable salt thereof.

38. The method of claim 33, wherein the compound is 2-[4-(2-carboxypropoxy)-3-cyanophenyl]-4-methyl-5-thiazolecarboxylic acid or a pharmaceutically acceptable salt thereof.

39. The method of claim 33, wherein the compound is 1-3-Cyano-4-(2,2-dimethylpropoxy)phenyl]-lH-pyrazole-4-carboxylic acid or a pharmaceutically acceptable salt thereof.

40. The method of claim 33, wherein the compound is pyrazolo [1,5-a]-1,3,5-triazin-4-(1H)-one, 8-[3-methoxy-4-(phenylsulfinyl)phenyl]-sodium salt (i).

41. The method of claim 33, wherein the compound is 3-(2-methyl-4-pyridyl)-5-cyano-4-isobutoxyphenyl)-1,2,4-triazole or a pharmaceutically acceptable salt thereof.

42. The method of claim 33, wherein the subject has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg.

43. A method of lowering blood pressure in a subject, the method comprising the step of:
administering to the subject a therapeutically effective amount of a compound or a pharmaceutically acceptable salt thereof wherein said compound comprises the formula: 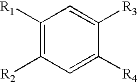
wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;
wherein R₃ and R4 are each independently a hydrogen or A, B, C or D as shown below: 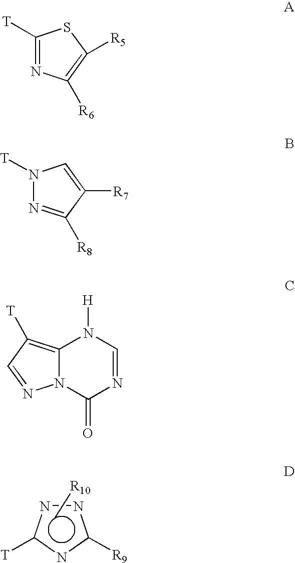
wherein T connects A, B, C or D to the aromatic ring shown above at R₁, R₂, R₃ or R₄.
wherein R₅ and R6 are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₇ and R8 are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₉ is an unsubstituted pyridyl group or a substituted pyridyl group; and
wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above.

44. The method of claim 43, wherein the compound is 2-[3-cyano-4-(2-methylpropoxy)phenyl]-4-methylthiazole-5-carboxylic acid or a pharmaceutically acceptable salt thereof.

45. The method of claim 43, wherein the compound is 2-[3-cyano-4-(3-hydroxy-2-methylpropoxy)phenyl]-4-methyl-5-thiazolecarboxylic acid or a pharmaceutically acceptable salt thereof.

46. The method of claim 43, wherein the compound is 2-[3-cyano-4-(2-hydroxy-2-methylpropoxy)phenyl]-4-methyl-5-thiazolecarboxylic acid or a pharmaceutically acceptable salt thereof.

47. The method of claim 43, wherein the compound is 2-(3-cyano-4-hydroxyphenyl)-4-methyl-5-thiazolecarboxylic acid or a pharmaceutically acceptable salt thereof.

48. The method of claim 43, wherein the compound is 2-[4-(2-carboxypropoxy)-3-cyanophenyl]-4-methyl-5-thiazolecarboxylic acid or a pharmaceutically acceptable salt thereof.

49. The method of claim 43, wherein the compound is 1-3-Cyano-4-(2,2-dimethylpropoxy)phenyl]-1H-pyrazole-4-carboxylic acid or a pharmaceutically acceptable salt thereof.

50. The method of claim 43, wherein the compound is pyrazolo [1,5-a]-1,3,5-triazin-4-(1H)-one, 8-[3-methoxy-4-(phenylsulfinyl)phenyl]-sodium salt (+).

51. The method of claim 43, wherein the compound is 3-(2-methyl-4-pyridyl)-5-cyano-4-isobutoxyphenyl)-1,2,4-triazole or a pharmaceutically acceptable salt thereof.

52. The method of claim 43, wherein the administration of the at least one compound lowers the systolic blood pressure, diastolic blood pressure, mean arterial pressure or a combination of the systolic blood pressure and diastolic blood pressure of the subject.

53. The method of claim 43, wherein the subject has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg.

54. The method of claim 43, wherein the subject has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg.

55. A method of decreasing elevated blood pressure in a subject, the method comprising the step of:
administering to the subject a therapeutically effective amount of a compound or a pharmaceutically acceptable salt thereof, wherein said compound comprises the formula: 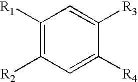
wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;
wherein R₃ and R₄ are each independently a hydrogen or A, B, C or D as shown below: 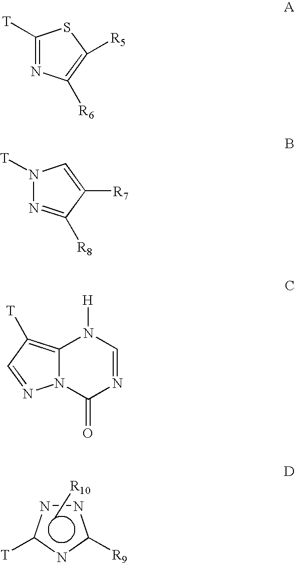
wherein T connects A, B, C or D to the aromatic ring shown above at R₁, R₂, R₃ or R₄.
wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₇ and R8 are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₉ is an unsubstituted pyridyl group or a substituted pyridyl group; and
wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above.

56. The method of claim 55, wherein the compound is 2-[3-cyano-4-(2-methylpropoxy)phenyl]-4-methylthiazole-5-carboxylic acid or a pharmaceutically acceptable salt thereof.

57. The method of claim 55, wherein the compound is 2-[3-cyano-4-(3-hydroxy-2-methylpropoxy)phenyl]-4-methyl-5-thiazolecarboxylic acid or a pharmaceutically acceptable salt thereof.

58. The method of claim 55, wherein the compound is 2-[3-cyano-4-(2-hydroxy-2-methylpropoxy)phenyl]-4-methyl-5-thiazolecarboxylic acid or a pharmaceutically acceptable salt thereof.

59. The method of claim 55, wherein the compound is 2-(3-cyano-4-hydroxyphenyl)-4-methyl-5-thiazolecarboxylic acid or a pharmaceutically acceptable salt thereof.

60. The method of claim 55, wherein the compound is 2-[4-(2-carboxypropoxy)-3-cyanophenyl]-4-methyl-5-thiazolecarboxylic acid or a pharmaceutically acceptable salt thereof.

61. The method of claim 55, wherein the compound is 1-3-Cyano-4-(2,2-dimethylpropoxy)phenyl]-1H-pyrazole-4-carboxylic acid or a pharmaceutically acceptable salt thereof.

62. The method of claim 55, wherein the compound is pyrazolo [1,5-a]-1,3,5-triazin-4-(1H)-one, 8-[3-methoxy-4-(phenylsulfinyl)phenyl]-sodium salt (±).

63. The method of claim 55, wherein the compound is 3-(2-methyl-4-pyridyl)-5-cyano-4-isobutoxyphenyl)-1,2,4-triazole or a pharmaceutically acceptable salt thereof.

64. The method of claim 55, wherein the subject has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg.

65. The method of claim 55, wherein the subject has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg.

66. The method of claims 55, wherein the administration of the at least one compound lowers the systolic blood pressure, the diastolic blood pressure, the mean arterial pressure or a combination of the systolic blood pressure and the diastolic blood pressure of the subject.

67. A method of normalizing blood pressure in a subject having a history of hypertension, the method comprising the step of:
administering to the subject a therapeutically effective amount of a compound or a pharmaceutically acceptable salt thereof, wherein said compound comprises the formula: 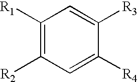
wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;
wherein R₃ and R4 are each independently a hydrogen or A, B, C or D as shown below: 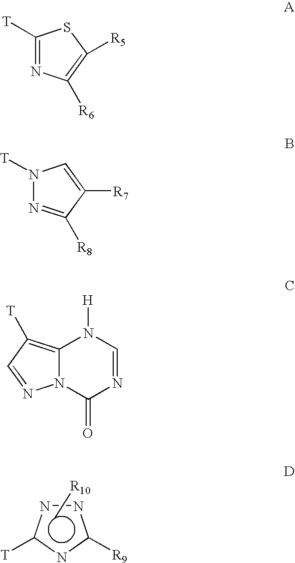
wherein T connects A, B, C or D to the aromatic ring shown above at R₁, R₂, R₃ or R₄.
wherein R₅ and R6 are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₉ is an unsubstituted pyridyl group or a substituted pyridyl group; and
wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above.

68. The method of claim 67, wherein the compound is 2-[3-cyano-4-(2-methylpropoxy)phenyl]-4-methylthiazole-5-carboxylic acid or a pharmaceutically acceptable salt thereof.

69. The method of claim 67, wherein the compound is 2-[3-cyano-4-(3-hydroxy-2-methylpropoxy)phenyl]-4-methyl-5-thiazolecarboxylic acid or a pharmaceutically acceptable salt thereof.

70. The method of claim 67, wherein the compound is 2-[3-cyano-4-(2-hydroxy-2-methylpropoxy)phenyl]-4-methyl-5-thiazolecarboxylic acid or a pharmaceutically acceptable salt thereof.

71. The method of claim 67, wherein the compound is 2-(3-cyano-4-hydroxyphenyl)-4-methyl-5-thiazolecarboxylic acid or a pharmaceutically acceptable salt thereof.

72. The method of claim 67, wherein the compound is 2-[4-(2-carboxypropoxy)-3-cyanophenyl]-4-methyl-5-thiazolecarboxylic acid or a pharmaceutically acceptable salt thereof.

73. The method of claim 67, wherein the compound is 1-3-cyano-4-(2,2-dimethylpropoxy)phenyl]-1H-pyrazole-4-carboxylic acid or a pharmaceutically acceptable salt thereof.

74. The method of claim 67, wherein the compound is pyrazolo [1,5-a]-1,3,5-triazin-4-(1H)-one, 8-[3-methoxy-4-(phenylsulfinyl)phenyl]-sodium salt (±).

75. The method of claim 67, wherein the compound is 3-(2-methyl-4-pyridyl)-5-cyano-4-isobutoxyphenyl)-1,2,4-triazole or a pharmaceutically acceptable salt thereof.

76. The method of claim 67, wherein the administration of the at least one compound normalizes the systolic blood pressure, the diastolic blood pressure, the mean arterial pressure or a combination of the systolic blood pressure and diastolic blood pressure of the subject.

77. The method of claim 67, wherein the subject has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg.

78. The method of claim 67, wherein the subject has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg.

79. A method for treating pre-hypertension in a subject in need of treatment thereof, the method comprising the step of:
administering to the subject a therapeutically effective amount of a compound or a pharmaceutically acceptable salt thereof, wherein said compound comprises the formula: 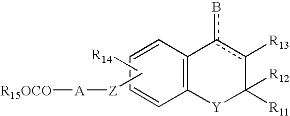
wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;
wherein R₁₃ is a hydrogen or a substituted or unsubstituted lower alkyl group;
wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R₁₇′, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R₁₇′ are each independently a hydrogen or a substituted or unsubstituted lower alkyl;
wherein R₁₅ is a hydrogen or a pharmaceutically active ester-forming group;
wherein A is a straight or branched hydrocarbon radical having one to five carbon atoms;
wherein B is a halogen, an oxygen, or a ethylenedithio;
wherein Y is an oxygen, a sulfur, a nitrogen or a substituted nitrogen;
wherein Z is an oxygen, a nitrogen or a substituted nitrogen; and
the dotted line refers to either a single bond, a double bond, or two single bonds.

80. The method of claim 79, wherein the subject has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg.

81. A method for treating hypertension in a subject in need of treatment thereof, the method comprising the step of:
administering to the subject a therapeutically effective amount of a compound or a pharmaceutically acceptable salt thereof, wherein said compound comprises the formula: 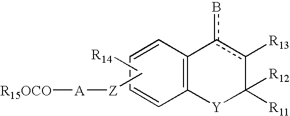
wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;
wherein R₁₃ is a hydrogen or a substituted or unsubstituted lower alkyl group;
wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R₁₇′, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R₁₇ are each independently a hydrogen or a substituted or unsubstituted lower alkyl;
wherein R₁₅ is a hydrogen or a pharmaceutically active ester-forming group;
wherein A is a straight or branched hydrocarbon radical having one to five carbon atoms;
wherein B is a halogen, an oxygen, or a ethylenedithio;
wherein Y is an oxygen, a sulfur, a nitrogen or a substituted nitrogen;
wherein Z is an oxygen, a nitrogen or a substituted nitrogen; and
the dotted line refers to either a single bond, a double bond, or two single bonds.

82. The method of claim 81, wherein the subject has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg.

83. A method of lowering blood pressure in a subject, the method comprising the step of:
administering to the subject a therapeutically effective amount of a compound or a pharmaceutically acceptable salt thereof wherein said compound comprises the formula: 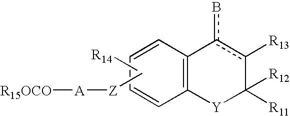
wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;
wherein R₁₃ is a hydrogen or a substituted or unsubstituted lower alkyl group;
wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R₇′, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R₁₇′ are each independently a hydrogen or a substituted or unsubstituted lower alkyl;
wherein R₁₅ is a hydrogen or a pharmaceutically active ester-forming group;
wherein A is a straight or branched hydrocarbon radical having one to five carbon atoms;
wherein B is a halogen, an oxygen, or a ethylenedithio;
wherein Y is an oxygen, a sulfur, a nitrogen or a substituted nitrogen;
wherein Z is an oxygen, a nitrogen or a substituted nitrogen; and
the dotted line refers to either a single bond, a double bond, or two single bonds.

84. The method of claim 83, wherein the administration of the at least one compound lowers the systolic blood pressure, diastolic blood pressure, mean arterial pressure or a combination of the systolic blood pressure and diastolic blood pressure of the subject.

85. The method of claim 83, wherein the subject has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg.

86. The method of claim 83, wherein the subject has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg.

87. A method of decreasing elevated blood pressure in a subject, the method comprising the step of:
administering to the subject a therapeutically effective amount of a compound or a pharmaceutically acceptable salt thereof, wherein said compound comprises the formula: 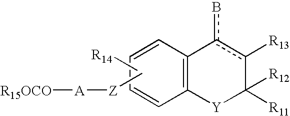
wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;
wherein R₁₃ is a hydrogen or a substituted or unsubstituted lower alkyl group;
wherein R₁₄ is one or two radicals selected from a group consisting of a hydrogen, a halogen, a nitro group, a substituted or unsubstituted lower alkyl, a substituted or unsubstituted phenyl, —OR₁₆ and -SO₂NR₁₇R₁₇′, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R₁₇′ are each independently a hydrogen or a substituted or unsubstituted lower alkyl;
wherein R₁₅ is a hydrogen or a pharmaceutically active ester-forming group;
wherein A is a straight or branched hydrocarbon radical having one to five carbon atoms;
wherein B is a halogen, an oxygen, or a ethylenedithio;
wherein Y is an oxygen, a sulfur, a nitrogen or a substituted nitrogen;
wherein Z is an oxygen, a nitrogen or a substituted nitrogen; and
the dotted line refers to either a single bond, a double bond, or two single bonds.

88. The method of claim 87, wherein the subject has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg.

89. The method of claim 87, wherein the subject has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg.

90. The method of claims 87, wherein the administration of the at least one compound lowers the systolic blood pressure, the diastolic blood pressure, the mean arterial pressure or a combination of the systolic blood pressure and the diastolic blood pressure of the subject.

91. A method of normalizing blood pressure in a subject having a history of hypertension, the method comprising the step of:
administering to the subject a therapeutically effective amount of a compound or a pharmaceutically acceptable salt thereof, wherein said compound comprises the formula: 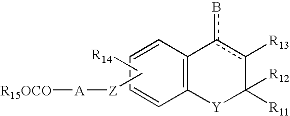
wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;
wherein R₁₃ is a hydrogen or a substituted or unsubstituted lower alkyl group;
wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R₁₇′, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R₁₇ are each independently a hydrogen or a substituted or unsubstituted lower alkyl;
wherein R₁₅ is a hydrogen or a pharmaceutically active ester-forming group;
wherein A is a straight or branched hydrocarbon radical having one to five carbon atoms;
wherein B is a halogen, an oxygen, or a ethylenedithio;
wherein Y is an oxygen, a sulfur, a nitrogen or a substituted nitrogen;
wherein Z is an oxygen, a nitrogen or a substituted nitrogen; and
the dotted line refers to either a single bond, a double bond, or two single bonds.

92. The method of claim 91, wherein the administration of the at least one compound normalizes the systolic blood pressure, the diastolic blood pressure, the mean arterial pressure or a combination of the systolic blood pressure and diastolic blood pressure of the subject.

93. The method of claim 91, wherein the subject has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg.

94. The method of claim 91, wherein the subject has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg.

95. The method of claims 1, 4, 7, 12 or 18, further comprising administering to the subject a therapeutically effective amount of at least one antihypertensive compound with the at least one xanthine oxidoreductase inhibitor or a pharmaceutically acceptable salt thereof.

96. The method of claims 23, 33, 43, 55 or 67, further comprising administering to the subject a therapeutically effective amount of at least one antihypertensive compound with the at least one compound or a pharmaceutically acceptable salt thereof.

97. The method of claims 79, 81, 83, 87 or 91, further comprising administering to the subject a therapeutically effective amount of at least one antihypertensive compound with the at least one compound or a pharmaceutically acceptable salt thereof.
